(19) United States
(12) Patent Application Publication
(10) Pub. No.: US 2011/0295038 A1
(43) Pub. Date: Dec. 1, 2011

(54) Process for the Preparation of Substituted 1-aminomethyl-2-phenyl-cyclohexane Compounds

(75) Inventors: Joerg HOLENZ, Enhoerna (SE); Helmut Heinrich Buschmann, Aachen (DE)

(73) Assignee: Gruenenthal GmbH, Aachen (DE)

(21) Appl. No.: 13/117,834

(22) Filed: May 27, 2011

(30) Foreign Application Priority Data
May 28, 2010 (EP) EP 10 005 613.4

(51) Int. Cl. C07C 209/50; C07C 209/48; C07C 209/74; C07C 209/78

(52) U.S. Cl. 564/414; 564/443

(57) ABSTRACT

A process for the preparation of substituted 1-aminomethyl-2-phenyl-cyclohexane compounds.

### BACKGROUND OF THE INVENTION

The present invention relates to a process for the preparation of substituted 1-aminomethyl-2-phenyl-cyclohexane compounds.

A class of active ingredients having excellent analgesic effectiveness and very good tolerability are substituted 1-aminomethyl-2-phenyl-cyclohexane compounds such as for example 3-(2-((dimethylamino)methyl)cyclohexyl)phenol as depicted below which are inter alia known from EP 0 753 506 B1.

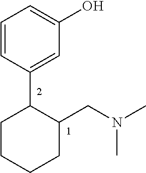

These compounds are conventionally prepared via a multi-step synthesis including a Mannich reaction as one of the key steps as it is exemplarily depicted below, e.g. for the synthesis of 3-(2-((dimethylamino)methyl)cyclohexyl)phenol

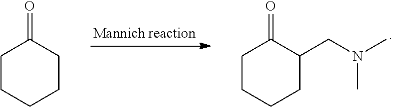

### SUMMARY OF THE INVENTION

An object of the present invention was to provide an alternative process which allows for the preparation of substituted 1-aminomethyl-2-phenyl-cyclohexane compounds.

A further object of the present invention was to provide such a process that has advantages over conventional processes for the preparation of substituted 1-aminomethyl-2-phenyl-cyclohexane compounds, in particular with respect to higher conversions and yields, flexibility, reducing the number of reaction steps, i.e. a shorter route, environmentally acceptable conditions, influence of stereoselectivity such as diastereoselectivity in a targeted manner and at least partial suppression of the formation of undesired side-product and/or undesired stereoisomers, in particular undesired diastereomers.

These and other objects have been achieved by the present invention as described and claimed hereinafter, i.e. by providing a process for the preparation of a compound according to formula (I), optionally in the form of one of its pure stereoisomers, in particular an enantiomer or diastereomer, a racemate or in form of a mixture of its stereoisomers, in particular enantiomers and/or diastereomers in any mixing ratio, or a physiologically acceptable acid addition salt thereof,

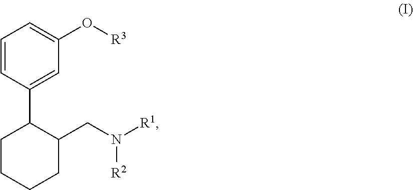

wherein
R¹, R² and R³ are independently of another selected from the group consisting of H and a C₁₋₄-aliphatic residue,
according to alternative A comprising the steps of:
   (a-I) hydrogenation of a compound according to formula (A-I), wherein R¹, R² and R³ have the above defined meanings,

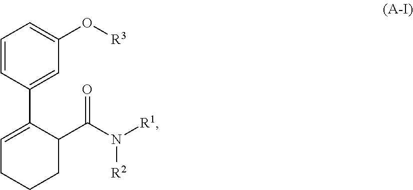

to a compound according to formula (A-II), wherein R¹, R² and R³ have the above defined meanings,

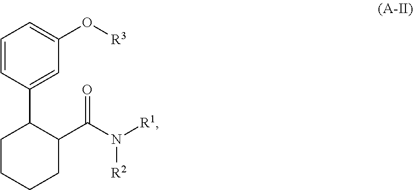

(a-II) reduction of a compound according to formula (A-II) to a compound according to formula (I),
(a-III) optionally converting the compound according to formula (I) into a physiologically acceptable acid addition salt thereof,
or according to alternative B comprising the steps of:
   (b-I) converting a compound according to formula (B-I), wherein R¹, R² and R³ have the above defined meanings, preferably wherein R¹ and R² have the above defined meanings and R³ is a C₁₋₄-aliphatic residue,

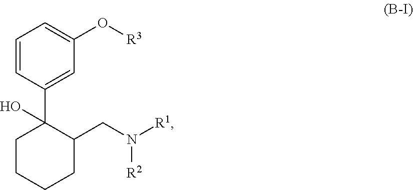

into a compound according to formula (B-II), wherein R¹, R² and R³ have the above defined meanings, preferably wherein R¹ and R² have the above defined meanings and R³ is a C₁₋₄-aliphatic residue

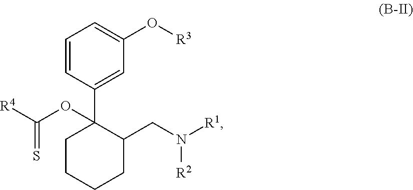

and R⁴ denotes (Y)ₙ—R⁵, wherein n denotes 0 or 1, Y denotes S, O, NH or an N(C₁₋₄-aliphatic residue) and R⁵ is selected from the group consisting of C₁₋₈-aliphatic residues, C₃₋₈-cycloaliphatic residues, aryl, heteroaryl, C₁₋₄-alkylene-C₃₋₈-cycloaliphatic residues, C₁₋₄-alkylene-aryl and C₁₋₄-alkylene-heteroaryl,
(b-II) converting a compound according to formula (B-II) into a compound according to formula (I), wherein R¹, R² and R³ have the above defined meanings, preferably wherein R¹ and R² have the above defined meanings and R³ is a C₁₋₄-aliphatic residue,
(b-III) optionally converting the compound according to formula (I) into a physiologically acceptable acid addition salt thereof,
or according to alternative C comprising the steps of:
   (c-I) hydrogenation of a compound according to formula (C-I), wherein R³ has the above defined meaning

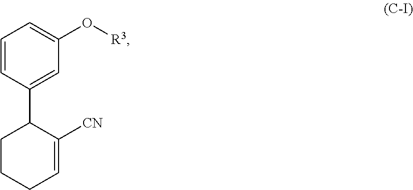

to a compound according to formula (C-II), wherein R³ has the above defined meaning,

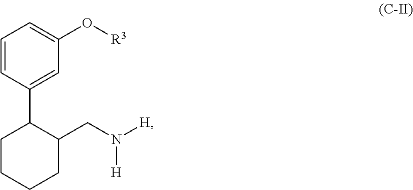

(c-II) optionally converting the thus obtained compound of formula (C-II) into a physiologically acceptable acid addition salt thereof
or
(c-III) optionally converting the thus obtained compound of formula (C-II) into a compound according to formula (I) and optionally converting the thus obtained compound according to formula (I) into a physiologically acceptable acid addition salt thereof,
or according to alternative D comprising the step of:
   (d-I) reacting a magnesium halide formed from a compound according to formula (D-I), wherein Hal is a halogen atom and wherein R³ has the above defined meaning, preferably wherein R³ denotes a C₁₋₄-aliphatic residue, with a compound according to formula (D-II), wherein Hal is a halogen atom and wherein R¹ and R² have the above defined meanings,

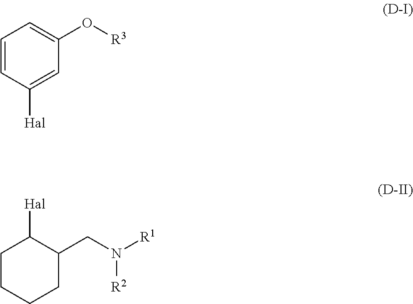

to a compound according to formula (I), wherein R¹, R² and R³ have the above defined meanings, preferably wherein R¹ and R² have the above defined meanings and R³ is a C₁₋₄-aliphatic residue, and
(d-II) optionally converting the compound according to formula (I) into a physiologically acceptable acid addition salt thereof.

It has been surprisingly found that by the process of the invention high conversions and yields can be achieved via a short reaction route and that the stereoselectivity, in particular diastereoselectivity can be influenced in a targeted manner by the choice of the reaction conditions and substrates. In particular, it has been surprisingly found that by the process of the invention the stereocenters may be established via substrate control with almost exclusive formation of the desired diastereomer(s), thus avoiding elaborate purification or resolution steps to separate stereoisomers and costly chiral reagents, catalysts or ligands. The process of the invention does not require a Mannich reaction to be performed. In case of alternative A, a particular advantage is the presence of a carbonyl group in the intermediate products such as (A-I) or (A-II), which allows the performance of an isomerization and/or epimerization reaction by abstracting the acidic hydrogen atom at the carbon atom bound to the carbonyl group, and thus allows the synthesis of specific stereoisomers, in particular diastereomers in a targeted manner.

As used herein, the terms “C₁₋₄-aliphatic residue” and “C₁₋₈-aliphatic residue” refer to a saturated or unsaturated, linear or branched, acyclic and unsubstituted hydrocarbon bearing 1 to 4, i.e. 1, 2, 3 or 4 or, respectively, 1 to 8, i.e. 1, 2, 3, 4, 5, 6, 7 or 8 carbon atoms. A C₁₋₄-aliphatic residue encompasses a C₁₋₄-alkyl group, a C₂₋₄-alkenyl group as well as a C₂₋₄-alkynyl group. A C₁₋₈-aliphatic residue encompasses a C₁₋₈-alkyl group, a C₂₋₈-alkenyl group as well as a C₂₋₈-alkynyl group. An alkenyl group has at least one C—C-double bond and an alkynyl has at least one C—C-triple bond. Examples of preferred C₁₋₄-aliphatic residues are methyl, ethyl, n-propyl, isopropyl, n-butyl, sec.-butyl, isobutyl, tert.-butyl, vinyl, allyl, butadienyl, ethynyl and propargyl. Examples of preferred C₁₋₈-aliphatic residues are methyl, ethyl, n-propyl, isopropyl, n-butyl, sec.-butyl, isobutyl, tert.-butyl, n-pentyl, neo-pentyl, isopentyl, n-hexyl, n-heptyl, n-octyl, vinyl, allyl, butenyl, butadienyl, ethynyl, propargyl and butynyl. A preferred “C₁₋₈-aliphatic residue” is a C₁₋₈-alkyl, more preferably a C₁₋₄-alkyl selected from the group consisting of methyl, ethyl, n-propyl, isopropyl, n-butyl, sec.-butyl, isobutyl and tert.-butyl. A preferred “C₁₋₄-aliphatic residue” is a C₁₋₄-alkyl, more preferably a C₁₋₄-alkyl selected from the group consisting of methyl, ethyl, n-propyl, isopropyl, n-butyl, sec.-butyl, isobutyl, tert.-butyl, even more preferably selected from the group consisting of methyl and ethyl. A particularly preferred C₁₋₄-aliphatic residue is a methyl group.

As used herein, the term “C₃₋₈-cycloaliphatic residue” refers to a saturated or unsaturated cyclic hydrocarbon bearing 3 to 8, i.e. 3, 4, 5, 6, 7, or 8 carbon atoms. A C₃₋₈-cycloaliphatic residue encompasses a C₃₋₈-cycloalkyl group and a C₃₋₈-cycloalkenyl group. A C₃₋₈-cycloalkenyl group has at least one C—C-double bond. A C₃₋₈-cycloaliphatic residue may be unsubstituted or mono- or polysubstituted with 1, 2, 3 or 4 substituents independently one another selected from the group consisting of OH, OC₁₋₄-alkyl, ═O, halogen, preferably F, Cl, Br, or I, NH₂, NH(C₁₋₄-alkyl), N(C₁₋₄-alkyl)₂, C₁₋₄-alkyl and phenyl. Examples of preferred C₃₋₈-cycloaliphatic residues are cyclopropyl, cyclobutyl, cyclopentyl, cyclohexyl, cycloheptyl, cyclooctyl, cyclopentenyl, cyclohexenyl, cycloheptenyl and cyclooctenyl.

As used herein, the term “aryl” refers to a cyclic aromatic hydrocarbon bearing 6 to 14 ring members, which may be condensed with an aryl group, a heteroaryl group or a C₃₋₈-cycloaliphatic residue. An aryl group may be unsubstituted or mono- or polysubstituted with 1, 2, 3 or 4 substituents independently one another selected from the group consisting of OH, OC₁₋₄-alkyl, ═O, halogen, preferably F, Cl, Br, or I, NH₂, NH(C₁₋₄-alkyl), N(C₁₋₄-alkyl)₂, C₁₋₄-alkyl and phenyl. Examples of preferred aryls are phenyl, 1-naphthyl and 2-naphthyl.

As used herein, the term “heteroaryl” refers to a cyclic aromatic ring bearing 5, 6 or 7 ring members containing at least one, optionally 2, 3 or 4 heteroatoms selected from the group consisting of N, O and S as ring member(s), which may be condensed with an aryl group, a heteroaryl group or a C₃₋₈-cycloaliphatic residue. A heteroaryl group may be unsubstituted or mono- or polysubstituted with 1, 2, 3 or 4 substituents independently one another selected from the group consisting of OH, OC₁₋₄-alkyl, ═O, halogen, preferably F, Cl, Br, or I, NH₂, NH(C₁₋₄-alkyl), N(C₁₋₄-alkyl)₂, C₁₋₄-alkyl and phenyl. Examples of preferred heteroaryls are furyl, pyrazolyl, pyridyl (2-pyridyl, 3-pyridyl, 4-pyridyl), pyrrolyl, pyridazinyl, pyrimidinyl, pyrazinyl and thienyl (thiophenyl).

As used herein, the term “C₁₋₄-alkylene” in connection with “C₃₋₈-cycloaliphatic residue”, “aryl” and “heteroaryl” refers to a C₃₋₈-cycloaliphatic residue, aryl or heteroaryl group which is bound to the corresponding greater structure such as in formula (B-II) by a C₁₋₄-alkylene group, i.e. a saturated or unsaturated, linear or branched, acyclic and unsubstituted hydrocarbon bearing 1 to 4, i.e. 1, 2, 3 or 4 carbon atoms. Preferred C₁₋₄-alkylene groups are selected from the group consisting of —CH₂—, —CH₂—CH₂—, —CH(CH₃)—, —CH₂—CH₂—CH₂—, and —CH(CH₃)—CH₂—.

As used herein, the term “physiologically acceptable acid addition salt” refers to an acid addition salt of a compound such as a compound according to formula (I) and at least one inorganic or organic acid, which are—in particular when administered to a human and/or a mammal—physiologically acceptable. Suitable physiologically acceptable acid addition salts include acid addition salts of inorganic acids, such as e.g. hydrogen chloride, hydrogen bromide and sulfuric acid, and salts of organic acids, such as methanesulfonic acid, fumaric acid, maleic acid, acetic acid, oxalic acid, succinic acid, malic acid, tartaric acid, mandelic acid, lactic acid, citric acid, glutaminic acid, acetylsalicylic acid, nicotinic acid, aminobenzoic acid, a-lipoic acid, hippuric acid and aspartic acid. The most preferred acid addition salt is a hydrochloride.

As used herein, the symbol

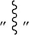

used in formulas throughout the present application such as for example in formula (C-I) refers to a single bond between a first carbon atom forming a double bound with a second carbon atom and a substituent, indicating that the substituent bonded to the first carbon atom may be either in trans- or in cis-position (or in (E)- or (Z)-position, respectively), with respect to the substituent(s) bound to the second carbon atom.

Preferably, the substituent groups R¹ and R² of the compound according to formula (I) prepared by the process of the invention are each independently selected from the group consisting of H, methyl, ethyl, n-propyl, isopropyl, n-butyl, sec.-butyl, isobutyl, tert.-butyl, more preferably independently of another selected from the group consisting of H and methyl, and the substituent group R³ of the compound according to formula (I) prepared by the process of the invention is preferably selected from the group consisting of H, methyl, ethyl, n-propyl, isopropyl, n-butyl, sec.-butyl, isobutyl, tert.-butyl, more preferably selected from the group consisting of H and methyl, even more preferably denotes H.

In a preferred embodiment of the present invention, the compound according to formula (I) is a compound according to formula (Ib) as depicted below, wherein R¹ and R² are independently of another selected from the group consisting of H and a C₁₋₄-aliphatic residue.

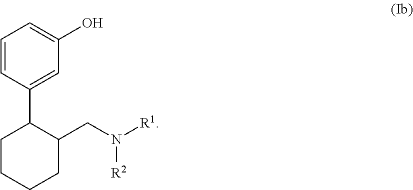

The present invention also relates to a process for the preparation of the stereoisomers of the compound of formula (I), such as enantiomers or diastereomers.

In another preferred embodiment of the present invention, the compound of formula (I) prepared by the process of the invention is a compound according to the formulae (I-1), (I-2), (I-3) and/or (I-4) and any mixture in any mixing ratio thereof, optionally in the form of a physiologically acceptable acid addition salt, wherein R¹, R² and R³ have one of the above defined meanings. Preferably, R³ denotes H in each of the formulae (I-1), (I-2), (I-3) and (I-4).

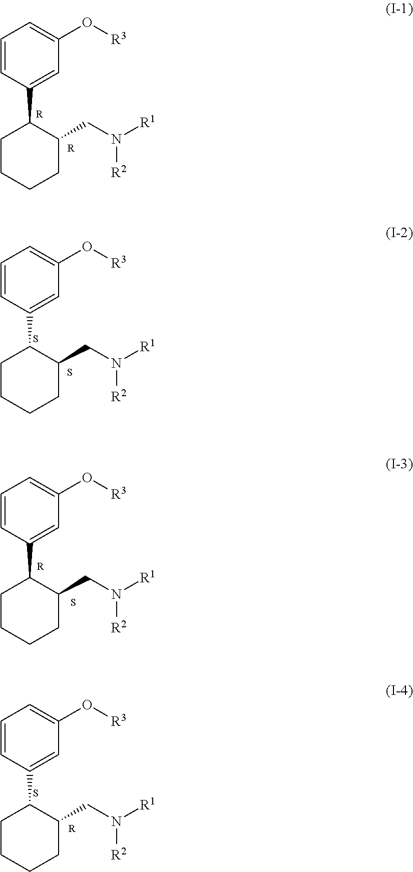

Preferred compounds prepared by the process of the invention are compounds according to formulas (I-1) and/or (I-2) and any mixture in any mixing ratio thereof, optionally in the form of a physiologically acceptable acid addition salt, more preferably a compound according to formula (I-1), optionally in the form of a physiologically acceptable acid addition salt.

In particular, the compound of formula (I) or (Ib) prepared by the process of the invention is a compound according to the formulae (I-1a), (I-2a), (I-3a) and/or (I-4a) and any mixture in any mixing ratio thereof, optionally in the form of a physiologically acceptable acid addition salt.

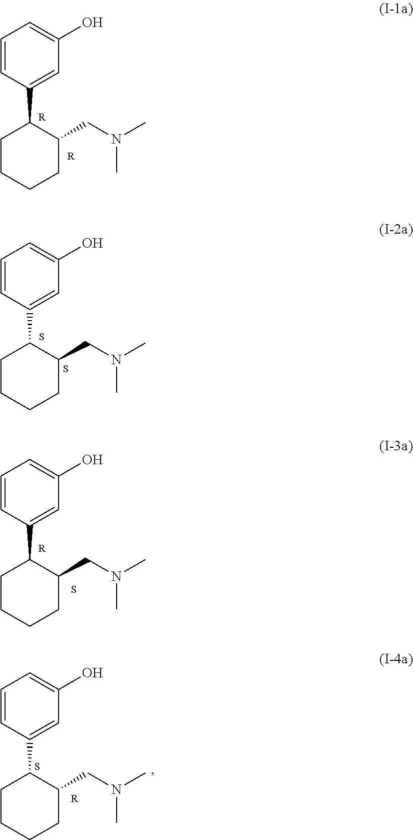

namely
3-((1R,2R)-2-((dimethylamino)methyl)cyclohexyl)phenol (I-1a),
3-((1S,2S)-2-((dimethylamino)methyl)cyclohexyl)phenol (I-2a),
3-((1R,2S)-2-((dimethylamino)methyl)cyclohexyl)phenol (I-3a),
3-((1S,2R)-2-((dimethylamino)methyl)cyclohexyl)phenol (I-4a),
and any mixture in any mixing ratio thereof, optionally in the form of a physiologically acceptable acid addition salt. Particularly preferred compounds prepared by the process of the invention are compounds according to formulae (I-1a) and/or (I-2a), and any mixture in any mixing ratio thereof, optionally in the form of a physiologically acceptable acid addition salt. The most preferred compound prepared by the process of the invention is a compound according to formula (I-1a), optionally in the form of a physiologically acceptable acid addition salt. The compound according to formula (I-1a) is known as Faxeladol.

Alternatives A, B and C of the process of the invention are particularly preferred.

### Process According to Alternative A

The process of the invention according to alternative A comprises at least the steps (a-I) and (a-II), i.e. hydrogenation of a compound according to formula (A-I) to a compound according to formula (A-II) (step a-I), wherein R¹, R² and R³ in each case have one of the above defined meanings, and reducing a compound according to formula (A-II) to a compound according to formula (I), wherein R¹, R² and R³ in each case have one of the above defined meanings (step a-II) as depicted in the following Scheme A1:

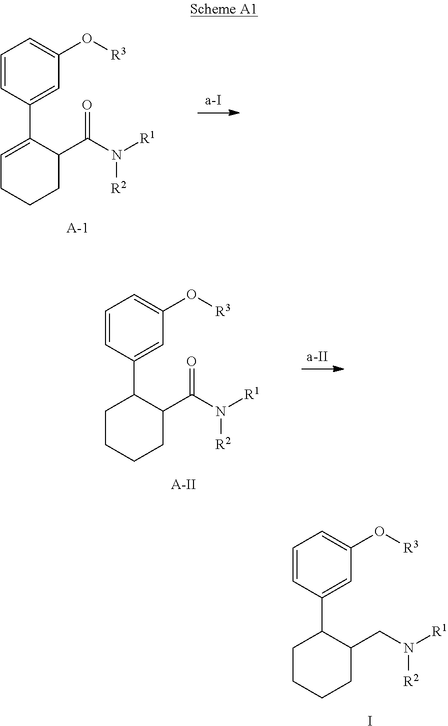

Step (a-I)

Preferably, the hydrogenation step (a-I) of the process of the invention according to alternative A is effected via heterogeneous or homogeneous catalysis, in each case in the presence of hydrogen. The hydrogen employed is preferably in gaseous form or at least part of it is dissolved in a liquid phase. In particular, the hydrogenation step (a-I) of the process of the invention according to alternative A is effected via heterogeneous catalysis.

In a preferred embodiment of the present invention, the compound according to formula (A-II) is not isolated, i.e. a compound according to formula (A-I) can be directly transformed into a compound according to formula (I) in one step, i.e. steps (a-I) and (a-II) can be carried out in one step (a-I-II) by performing a hydrogenation reaction in combination with a reducing step.

The term catalyst within the context of the present invention includes both catalytically active materials themselves and inert materials that are provided with a catalytically active material. Accordingly, the catalytically active material can, for example, be applied to an inert carrier or can be present in a mixture with an inert material. There come into consideration as inert carrier or inert material, for example, carbon and other materials known to the person skilled in the art.

If a homogeneous catalyst in hydrogenation step (a-I) according to alternative A of the process of the invention is employed, said homogeneous catalyst is preferably a transition metal complex of rhodium, iridium or ruthenium, particularly preferably a transition metal complex of rhodium or iridium, more particularly a transition metal complex of rhodium with diphosphine ligands.

Diphosphine ligands which can preferably be employed are, for example known from the following literature references: a) H. Brunner, W. Zettlmeier, Handbook of Enantioselective Catalysis. VCH Weinheim, 1993, vol. 2; b) R. Noyori et al. in Catalytic Asymmetric Synthesis Second Edition (I. Ojima, Ed.), Wiley-VCH, Weinheim, 2000; c) E. N. Jacobsen, A. Pfaltz, H. Yamamoto (Eds.), Comprehensive Asymmetric Catalysis Vol I-III, Springer Berlin, 1999, and the references cited therein.

Particularly preferably the catalyst is chosen from the group consisting of rhodium (−)-DIPAMP [(R,R)-(−)-1,2-Bis[(2-methoxyphenyl)(phenyl)phosphino]ethane], rhodium (+)-DIPAMP [(S,S)-(+)-1,2-Bis[(2-methoxyphenyl)(phenyl)phosphino]ethane], rhodium R-Solphos [R-(+)-N,N′-Dimethyl-7,7′-bis(diphenylphosphino)-3,3′,4,4′-tetrahydro-8,8′-bi-2H-1,4-benzoxazine] and rhodium S-Solphos [S-(−)-N,N′-Dimethyl-7,7′-bis(diphenylphosphino)-3,3′,4,4′-tetrahydro-8,8′-bi-2H-1,4-benzoxazine].

The reaction parameters for the homogeneous hydrogenation in step (a-I), such as, for example, pressure, temperature or reaction time, can vary over a wide range. Preferably, the temperature during the homogeneous hydrogenation in step (a-I) can be in each case from 0 to 250° C., particularly preferably from 5 to 100° C., very particularly preferably from 10 to 60° C. and most preferred from 15 to 25° C. The homogeneous hydrogenation in step (a-I) can preferably be carried out at reduced pressure, at normal pressure or at elevated pressure, preferably in the range from 0.01 to 300 bar. It is particularly preferred to carry out the reactions under pressure in a range of from 1 to 200 bar, in particular from 10 to 100 bar.

The reaction time can vary depending on various parameters, such as, for example, temperature, pressure, nature of the compound to be reacted or the properties of the catalyst, and can be determined for the process in question by the person skilled in the art using preliminary tests.

If a heterogeneous catalyst in hydrogenation step (a-I) according to alternative A of the process of the invention is employed, said heterogeneous catalyst preferably comprises one or more transition metals, which can preferably be selected from the group consisting of Cu, Ag, Au, Zn, Cd, Hg, V, Nb, Ta, Cr, Mo, W, Fe, Ru, Os, Co, Rh, Ir, Ni, Pd and Pt, more preferably from the group consisting of Ru, Rh, Pd, Pt and Ni, and in particular from the group consisting of Pd, Pt and Ni.

Heterogeneous catalysis within the context of the present invention means that the catalysts employed in heterogeneous catalysis are in each case present in the solid state of aggregation.

Preferably, heterogeneous catalysts according to the present invention can comprise one or more of the above-mentioned transition metals in the same or different oxidation states. It may also be preferable for the corresponding catalysts to contain one or more of the above-mentioned transition metals in two or more different oxidation states.

The preparation of heterogeneous catalysts doped with transition metals can be carried out by conventional processes known to persons skilled in the art.

In particular, a heterogeneous catalyst is employed in hydrogenation reaction step (a-I) in alternative A of the process of the invention. Preferred heterogeneous catalysts employed in this steps are independently of another selected from the group consisting of Raney nickel, palladium, palladium on carbon (1-10 wt. %, preferably 5 wt. %), platinum, platinum on carbon (1-10 wt. %, preferably 5 wt. %), ruthenium on carbon (1-10 wt. %, preferably 5 wt. %) and rhodium on carbon (1-10 wt. %, preferably 5 wt. %). Most preferred is palladium on carbon (1-10 wt. %, preferably 5 wt. %) as the catalyst for hydrogenation in step (a-I).

The compound according to formula (A-I) according to the process of the invention is preferably in liquid phase and to that end are preferably mixed with or dissolved in a reaction medium that is liquid under the particular reaction conditions. Examples of suitable reaction media employed in hydrogenation reactions are methanol, ethanol, isopropanol, n-butanol, n-propanol, toluene, n-heptane, n-hexane, n-pentane, acetic acid, ethyl acetate, formic acid, hydrochloric acid, hydrobromic acid, sulfuric acid and any mixtures thereof. More preferably ethanol is used as the reaction medium in step (a-I). Of course, it is also possible to use mixtures or multiphase systems comprising two or more of the above-mentioned liquids in the processes according to the present invention.

The reaction parameters for the hydrogenation reactions in step (a-I) such as, for example, pressure, temperature or reaction time, can independently of another vary over a wide range both. Preferably, the temperature during the heterogeneous hydrogenation in step (a-I) is in each case from 0 to 250° C., particularly preferably from 15 to 180° C. and very particularly preferably from 15 to 30° C. The heterogeneous hydrogenation in step (a-I) can preferably be carried out at reduced pressure, at normal pressure or at elevated pressure, preferably in the range from 0.5 to 300 bar. It is particularly preferred to carry out the reactions under pressure in a range from 0.5 to 10 bar, in particular from 0.75 to 10 bar. The reaction time can vary in dependence on various parameters, such as, for example, temperature, pressure, nature of the compound to be reacted or the properties of the catalyst, and can be determined for the process in question by the person skilled in the art using preliminary tests.

The continuous removal of samples in order to monitor the reaction, for example by means of gas chromatography (GC) methods, is also possible, optionally in combination with regulation of the corresponding process parameters.

The total amount of the heterogeneous catalyst(s) used depends on various factors, such as, for example, the ratio of the catalytically active component to any inert material present, or the nature of the surface of the catalyst(s). The optimal amount of catalyst(s) for a particular reaction can be determined by the person skilled in the art using preliminary tests.

The particular compound of formula (A-II) obtained in step (a-I) can be isolated and/or purified by conventional methods known to a person skilled in the art.

Step (a-II)

Preferably, the reduction step (a-II) of the process of the invention according to alternative A is performed in the presence of at least one suitable reducing agent.

Any reducing agent suitable for the reduction of an amide group to an amine group may be employed. Preferably, the suitable reducing agent is at least one metal hydride, more preferably at least one metal hydride selected from the group consisting of lithium aluminium hydride (LAH), sodium borohydride, diisobutyl aluminium hydride (DIBAL), selectrides such as L-selectride, N-selectride and K-selectride, or the suitable reducing agent at least one borane such as borane-THF or the suitable reducing agent is hydrogen in combination with a catalyst, preferably in combination with a heterogeneous catalyst. Most preferred is lithium aluminium hydride.

Preferably, in step (a-II)—in addition to the reducing agent—at least one Lewis acid is employed in combination with the reducing agent. A particularly preferred Lewis acid is aluminium trichloride (AlCl₃).

Preferably, in step (a-II) the reducing agent, optionally in combination with a Lewis acid, is dissolved or suspended in a suitable reaction medium and then the compound according to formula (A-II), which is preferably dissolved in a suitable solvent, is added to the solution or suspension comprising the reducing agent and optionally the Lewis acid.

Suitable solvents for the compound according to formula (A-II) are preferably selected from the group consisting of methanol, ethanol, isopropanol, 1,4-dioxane, tetrahydrofuran (THF) and any mixtures in any ratio thereof. A particularly preferred solvent is THF.

Suitable reaction media for dissolving or suspending the reducing agent and optionally the Lewis acid are preferably selected from the group consisting of methanol, ethanol, n-propanol, isopropanol, 1,4-dioxane, tetrahydrofuran (THF) and any mixtures in any ratio thereof. A particularly preferred reaction medium is THF.

The reaction parameters for the reduction in step (a-II) such as, for example, pressure, temperature or reaction time, can independently of one another vary over a wide range both. Preferably, the temperature in step (a-II) is in each case from 0 to 250° C., particularly preferably from 15 to 180° C. and very particularly preferably from 15 to 80° C. The reduction in step (a-II) can preferably be carried out at reduced pressure, at normal pressure or at elevated pressure, preferably in the range from 0.5 to 300 bar, if hydrogen in combination with a catalyst is employed as reducing agent. It is particularly preferred to carry out the reactions under pressure in a range from 0.5 to 10 bar, in particular from 0.75 to 5 bar, if hydrogen in combination with a catalyst is employed as reducing agent. The reaction time can vary in dependence on various parameters, such as, for example, temperature, pressure, nature of the compound to be reacted or the properties of the catalyst, and can be determined for the process in question by the person skilled in the art using preliminary tests.

The particular compound of formula (I) obtained in step (a-II) can be isolated and/or purified by conventional methods known to the person skilled in the art.

Step (a-III)

Optionally, the compound according to formula (I) may be converted into a physiologically acceptable acid addition salt thereof. The conversion of a compound according to formula (I) into a corresponding acid addition salt via reaction with a suitable acid or a suitable acid addition salt forming agent may be effected in a manner well known to those skilled in the art, e.g. by dissolving a compound according to formula (I) in at least one suitable solvent, preferably at least one solvent selected from the group consisting of acetone, benzene, n-butanol, 2-butanone, tert.-butyl methylether, chloroform, cyclohexane, diethyl ether, 1,4-dioxane, diisopropyl ether, alkyl acetates, e.g. ethyl acetate, ethanol, n-hexane, n-heptane, isopropanol, methanol, methylene chloride (dichloromethane), n-pentane, petrol ether, n-propanol, tetrahydrofuran, toluene, and any mixture in any mixing ratio thereof, and subsequent addition of at least one suitable acid or at least one acid addition salt forming agent. Preferably, the solvent employed for dissolving a compound according to formula (I) is a solvent in which the resulting acid addition salt of a compound according to formula (I) is not soluble.

The precipitation and/or crystallization of the acid addition salt may preferably be initiated and/or improved by cooling the corresponding reaction mixture and optionally partial evaporation of the solvent(s) under reduced pressure. The precipitate may then be filtered off, optionally washed with a suitable solvent, and if necessary further purified by recrystallization.

The salt formation may preferably be effected in a suitable solvent including diethyl ether, diisopropyl ether, dichloromethane, alkyl acetates, e.g. ethyl acetate, acetone, 2-butanone or any mixture thereof. Preferably a reaction with trimethylchlorosilane (trimethylsilylchloride) as acid addition salt forming agent in a suitable solvent may be used for the preparation of the corresponding hydrochloride addition salt.

Step (a-IV)

In a preferred embodiment of the process of the invention, alternative A further comprises a step (a-IV), wherein a compound according to formula (A-0), wherein R¹, R² and R³ have one of the above defined meanings, is subjected to a dehydration reaction to obtain the compound according to formula (A-I),

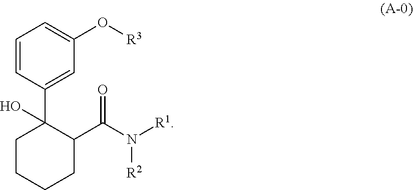

In Scheme A2 step (a-IV) is depicted below.

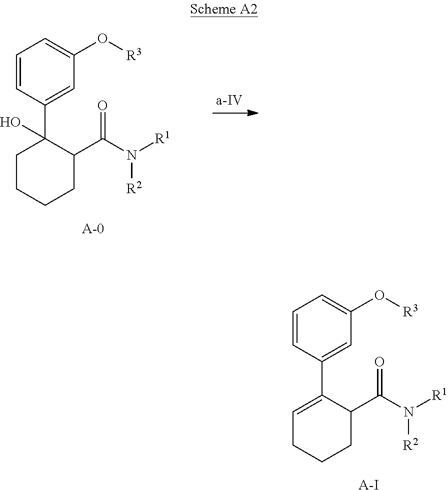

The dehydration step (a-IV) is preferably acid-catalyzed or acid-promoted, i.e. performed in the presence of an acid in a catalytically effective or at least stoichiometric amount. Preferably the acid is selected from the group consisting of formic acid, hydrochloric acid, acetic acid, sulfuric acid, hydrobromic acid, methanesulfonic acid or any mixture thereof. It is preferable if the acid is employed in a high concentration. Particularly preferably, hydrochloric acid and/or hydrobromic acid are employed. Preferably, the concentration of the hydrochloric acid or the hydrobromic acid is >20%, more preferably >30%, particularly preferably >35% by weight. Alternatively, the acid can also be used in gaseous form.

The compound of general formula (A-0) used in step (a-IV) according to the present invention is preferably in liquid phase and to that end is preferably mixed with or dissolved in a reaction medium that is liquid under the particular reaction conditions.

Examples of suitable reaction media include water, acetic acid, formic acid, toluene, hydrochloric acid, sulfuric acid, hydrobromic acid, methanesulfonic acid or any mixture thereof. Of course, it is also possible to use mixtures or multiphase systems comprising two or more of the above-mentioned liquids in the processes according to the present invention.

The reaction parameters for step (a-IV), such as, for example, pressure, temperature or reaction time, can vary over a wide range. It is preferable if the reaction temperature in step (a-IV) is between 15 and 100° C., particularly preferably between 18 and 80° C., more particularly preferably between 20 and 60° C. The dehydration step (a-IV) can preferably be carried out at reduced pressure, at normal pressure or at elevated pressure, preferably in the range from 0.01 to 300 bar. It is particularly preferred to carry out the reactions under pressure in a range from 0.5 to 5 bar, in particular from 0.5 to 1.5 bar.

The reaction time can vary in dependence on various parameters, such as, for example, temperature, pressure, nature of the compound to be reacted or the properties of the catalyst, and can be determined for the process in question by the person skilled in the art using preliminary tests. It is preferable if the reaction time of step (a-IV) is between 2 and 25 hours, particularly preferably between 3 and 22 hours, more particularly preferably between 4 and 20 hours.

The continuous removal of samples in order to monitor the reaction, for example by means of gas chromatographic (GC) methods, is also possible, optionally in combination with regulation of the corresponding process parameters.

The particular compound of general formula (A-I) obtained can be isolated and/or purified by conventional methods known to the person skilled in the art.

Alternatively, the dehydration step (a-IV) can also be carried out in the presence of at least one acidic catalyst, which can preferably be selected from the group consisting of ion-exchange resins, zeolites, heteropoly acids, phosphates, sulfates and optionally mixed metal oxides.

Preferably, the temperature for step (a-IV) when using an acidic catalyst as describe above is in each case from 20 to 250° C., particularly preferably from 50 to 180° C. and very particularly preferably from 100 to 160° C. The ratio of acidic catalyst and compound of formula (A-0) is preferably in the range from 1:200 to 1:1, in particular from 1:4 to 1:2. After the dehydration, the catalyst can be separated from the reaction mixture in a simple manner, preferably by filtration. The particular compound of general formula (A-I) obtained be isolated and/or purified by conventional methods known to the person skilled in the art.

Alternatively, the dehydration step (a-IV) can also be carried out by subjecting a compound of general formula (A-0) to an excess of thionyl chloride, optionally in a reaction medium, preferably in a reaction medium selected from the group consisting of diethylether, tetrahydrofuran, toluene, 2-methyltetrahydrofuran, dioxane, tert-butyl-methylether and any mixture thereof, and subsequent heating of the thus obtained reaction mixture to 40° C. to 120° C., preferably to 80° C. to 120° C.

Step (a-V)

Preferably, the compound of formula (A-II) obtained in step (a-I) according to alternative A of the process of the invention is a compound according to formulae (PI-3) and/or (PI-4) or any mixture in any mixing ratio thereof, wherein R¹, R² and R³ have one of the above defined meanings.

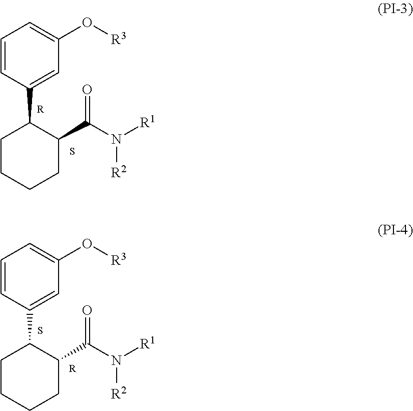

In a preferred embodiment, alternative A of the process of the invention comprises a step (a-V) prior to step (a-II), wherein the compound according to formula (PI-3) and/or (PI-4) or any mixture in any mixing ratio thereof is subjected to an isomerization reaction, preferably in the presence of a base, to obtain a compound according to formulae (PI-1) and/or (PI-2) or any mixture in any mixing ratio thereof as depicted in Scheme A3.

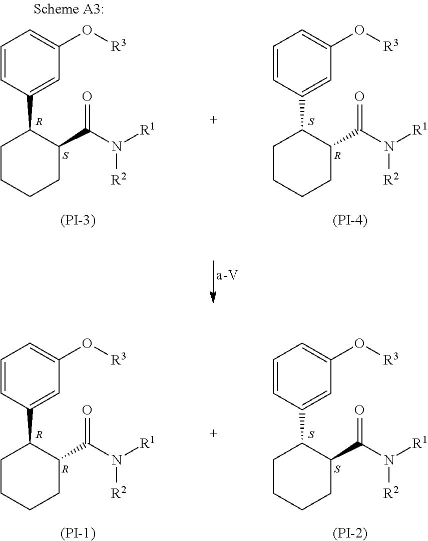

Suitable bases to be employed are any bases which are able to abstract the acidic proton of the carbon atom bound to the carbon atom of the carbonyl group of the compound according to formulae (PI-3) and (PI-4). Preferably, suitable bases which may be employed in step (a-V) are selected from the group consisting of 1,8-diazabicyclo[5.4.0]undec-7-ene (DBU), sodium hydride (NaH), potassium hydride, sodium hydroxide (NaOH), potassium hydroxide (KOH), amines, preferably tertiary amines, more preferably N(C₁₋₄-alkyl)₃, even more preferably triethylamine, sodium methanolate, potassium tert-butylate (KOtBu) and mixtures of two of any of the aforementioned bases in any mixing ratio. The most preferred bases are selected from the group consisting of potassium tert-butylate, potassium hydroxide and sodium hydride. In particular, potassium tert-butylate is employed as a base.

Suitable reaction media for the conversion of a compound according to formulae (PI-3) and/or (PI-4) or any mixture in any mixing ratio thereof into a compound to formulae (PI-1) and/or (PI-2) or any mixture in any mixing ratio thereof are preferably selected from the group consisting of acetone, benzene, n-butanol, 2-butanone, tert.-butyl methylether, chloroform, cyclohexane, diethyl ether, 1,4-dioxane, diisopropyl ether, alkyl acetates, e.g. ethyl acetate, ethanol, n-hexane, n-heptane, isopropanol, methanol, methylene chloride (dichloromethane), n-pentane, petrol ether, n-propanol, tetrahydrofuran, toluene, and any mixture in any mixing ratio thereof. Preferably, THF is used as reaction medium.

The reaction parameters for step (a-V), such as, for example, pressure, temperature or reaction time, can vary over a wide range. It is preferable if the reaction temperature in step (a-V) is between 15 and 100° C., particularly preferably between 18 and 80° C. Preferably, step (a-V) is carried out at reduced pressure, at normal pressure or at elevated pressure, preferably in the range from 0.01 to 300 bar. It is particularly preferred to carry out the reactions under pressure in a range from 0.5 to 5 bar, in particular from 0.5 to 1.5 bar.

The reaction time can vary in dependence on various parameters, such as, for example, temperature, pressure, nature of the compound to be reacted and can be determined for the process in question by the person skilled in the art using preliminary tests. It is preferable if the reaction time of step (a-V) is between 2 and 25 hours, particularly preferably between 3 and 22 hours, more particularly preferably between 4 and 20 hours.

The particular compound of general formulae (PI-1) and/or (PI-2) or any mixture in any mixing ratio thereof obtained can be isolated and/or purified by conventional methods known to the person skilled in the art.

In a preferred embodiment of alternative A of the process of the invention, a compound of general formula (PI-1) and/or (PI-2) or any mixture in any mixing ratio thereof as obtained via step (a-V) or a compound of general formula (PI-3) and/or (PI-4) or any mixture in any mixing ratio thereof as obtained via step (a-I) is employed as starting material in the reduction step (a-II) yielding a compound according to formulae (I-1) and/or (I-2) or any mixture in any mixing ratio thereof (starting from (PI-1) and (PI-41) or a compound according to formulae (I-3) and/or (I-4) or any mixture in any mixing ratio thereof (starting from (PI-3) and (PI-4)) according to alternative A of the process of the invention as depicted below in Scheme A4, wherein R¹, R² and R³ in each case have one of the above defined meanings:

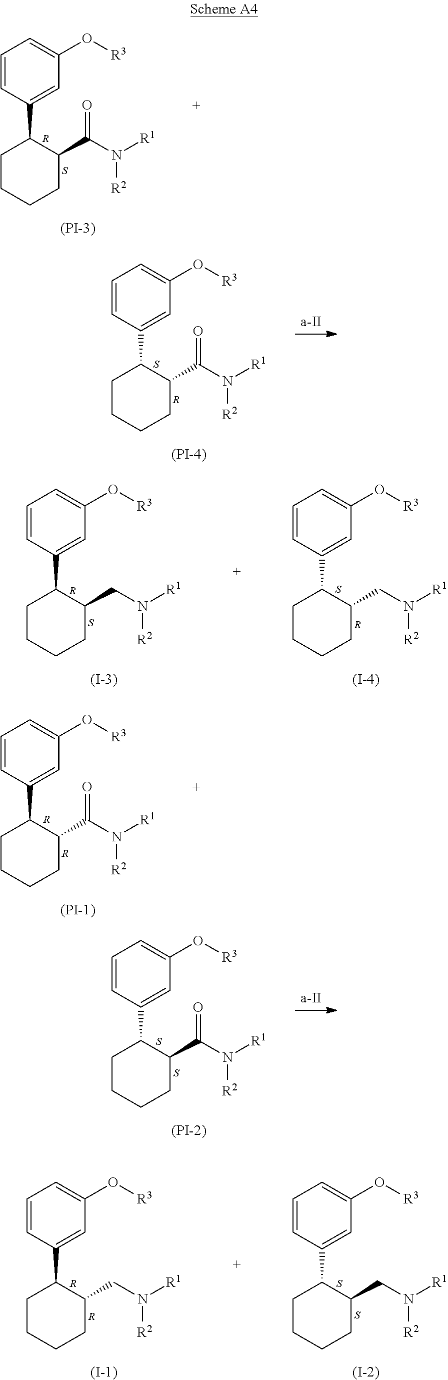

In a particularly preferred embodiment of the present invention, only a compound of general formulas (PI-1) and/or (PI-2) or any mixture in any mixing ratio thereof as obtained via step (a-V) is employed in step (a-II) of alternative A of the process of the invention yielding a compound according to formulas (I-1) and/or (I-2) or any mixture in any mixing ratio thereof, wherein R¹, R² and R³ have one of the above defined meanings.

Step (a-VI)

In a particularly preferred embodiment of the process of the invention, alternative A further comprises a deprotection step (a-VI), wherein one of the compounds according to formula (A-0), (A-I), (A-II) or (I), wherein R¹ and R² have in each case one of the above defined meanings and R³ in each case is ≠H, is deprotected to obtain a compound according to formula (Ib).

Preferably, the deprotection step (a-VI) is carried out by subjecting a compound according to formula (I) or (A-II), more preferably a compound according to formula (I), to said deprotection.

Preferably, at least one acid, preferably at least one acid selected from the group consisting of hydrobromic acid, hydrochloric acid and methanesulfonic acid is employed as deprotecting agent in step (a-VI). In case methanesulfonic acid is employed as acid a combination of methanesulfonic acid and methionine is preferably used as deprotecting agent. A combination of methanesulfonic acid and methionine is the most preferred deprotecting agent in step (a-VI). The deprotection step (a-VI) is preferably carried out in a reaction medium selected from the group consisting of diethylether, tetrahydrofuran, toluene, 2-methyltetrahydrofuran, dioxane, tert.-butyl methylether and any mixture thereof.

The reaction parameters for step (a-VI), such as, for example, pressure, temperature or reaction time, can vary over a wide range. It is preferable if the reaction temperature in step (a-VI) is between 15 and 100° C., particularly preferably between 18 and 80° C. Preferably, step (a-VI) is carried out at normal pressure.

The reaction time can vary depending on various parameters, such as, for example, temperature, pressure, nature of the compound to be reacted and can be determined for the process in question by the person skilled in the art using preliminary tests. It is preferable if the reaction time of step (a-VI) is between 2 and 25 hours, particularly preferably between 3 and 22 hours, more particularly preferably between 4 and 20 hours.

The compound according to formula (Ib) can be isolated and/or purified by conventional methods known to persons skilled in the art.

Optionally, the compound according to formula (Ib) may be converted into a physiologically acceptable acid addition salt thereof according to the procedure previously described in step (a-III).

Step (a-VII)

In a preferred embodiment of the process of the invention, alternative A further comprises a step (a-VII) for the preparation of a compound according to formula (A-0) as depicted in Scheme A5 below:

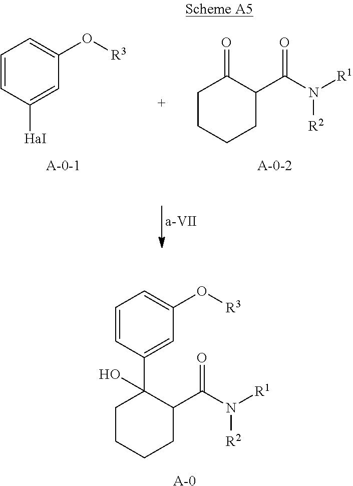

In step (a-VII) a magnesium halide, i.e. a Grignard reagent, is formed from a compound according to formula (A-0-1), wherein Hal is a halogen atom, preferably selected from the group consisting of Cl, Br and I, in particular Br, and magnesium in an inert reaction medium. Said Grignard reagent is then reacted with a compound according to formula (A-0-2) under Grignard conditions in an inert reaction medium, preferably in an organic ether, for example, selected from the group consisting of diethyl ether, tetrahydrofuran, 2-methyltetrahydrofuran, tert-butylmethyl ether or any mixture thereof to obtain a compound according to formula (A0).

The particular compound of general formula (A-0) obtained can be isolated and/or purified by conventional methods known to the person skilled in the art.

### Process According to Alternative B

The process of the invention according to alternative B comprises at least the step (b-I), i.e. a conversion of a compound according to formula (B-I), wherein R¹, R² and R³ have one of the above defined meanings, preferably wherein R¹ and R² have the above defined meanings and R³ is a C₁₋₄-aliphatic residue

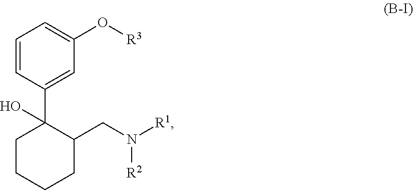

into a compound according to formula (B-II), wherein R¹, R² and R³ in each case have one of the above defined meanings, preferably wherein R¹ and R² have the above defined meanings and R³ is a C₁₋₄-aliphatic residue

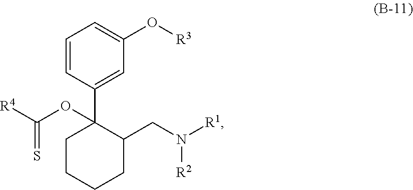

and R⁴ denotes (Y)ₙ—R⁵, wherein n denotes 0 or 1, Y denotes S, O, NH or an N(C₁₋₄-aliphatic residue) and R⁵ is selected from the group consisting of C₁₋₈-aliphatic residues, C₃₋₈-cycloaliphatic residues, aryl, heteroaryl, C₁₋₄-alkylene-C₃₋₈-cycloaliphatic residues, C₁₋₄-alkylene-aryl and C₁₋₄-alkylene-heteroaryl, and further at least the step (b-II) of converting a compound according to formula (B-II) into a compound according to formula (I), wherein R¹, R² and R³ have one of above defined meanings, preferably wherein R¹ and R² have the above defined meanings and R³ is a C₁₋₄-aliphatic residue, as depicted in the following Scheme B1:

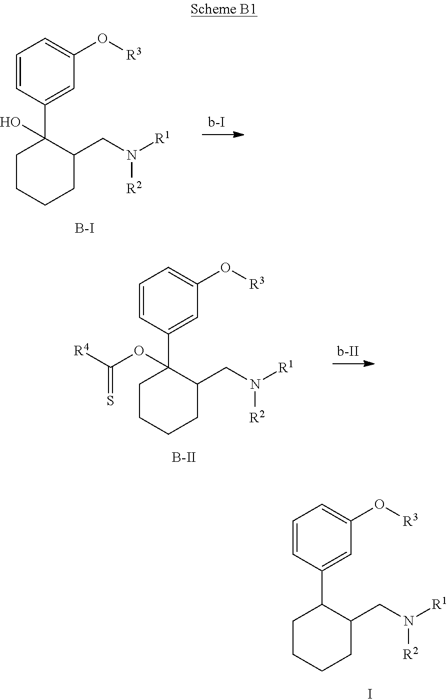

Optionally, if R³ is a C₁₋₄-aliphatic residue in the compound according to formula (B-II) or (I), the compound according to formula (B-II) or (I) may be subjected to a deprotection step (b-IV) as described below to obtain a compound according to formula (I), wherein R³ is ═H, i.e. a compound according to formula (Ib).

Step (b-I)

Preferably, in step (b-I) the compound according to formula (B-I) is reacted with a compound according to the formula R⁴—C(═S)-Hal, wherein Hal is preferably selected from the group consisting of Cl and Br, and wherein R⁴ has the above defined meaning.

In a preferred embodiment, the compound according to formula (B-I) is reacted with a compound according to the formula R⁴—C(═S)-Hal, wherein R⁴ denotes (Y)ₙ—R⁵, wherein n denotes 0 or 1, Y denotes O and R⁵ is selected from the group consisting of C₁₋₈-aliphatic residues, aryl and C₁₋₄-alkylene-aryl.

Preferably, in step (b-I) the compound according to formula (B-I) is dissolved or suspended in a suitable reaction medium and then the compound according to the formula R⁴—C(═S)-Hal, which may be optionally dissolved or suspended in a suitable reaction medium, is added to the solution or suspension comprising the compound according to formula (B-I).

Suitable reaction media for the compound according to formula (B-I) are preferably selected from the group consisting of diethyl ether 1,4-dioxane, tetrahydrofuran (THF) and any mixture in any ratio thereof. A particularly preferred reaction medium is THF.

Suitable reaction media for dissolving or suspending the compound according to the formula R⁴—C(═S)-Hal are preferably selected from the group consisting of diethyl ether 1,4-dioxane, tetrahydrofuran (THF) and any mixture in any ratio thereof. A particularly preferred reaction medium is THF.

The reaction parameters for the reduction in step (b-I) such as, for example, pressure, temperature or reaction time, can independently of another vary over a wide range. Preferably, the temperature in step (b-I) is in each case from 0 to 250° C., particularly preferably from 15 to 180° C. and very particularly preferably from 15 to 80° C. The reduction in step (b-I) can preferably be carried out at reduced pressure, at normal pressure or at elevated pressure, preferably in the range from 0.5 to 300 bar. It is particularly preferred to carry out the reactions under pressure in a range from 0.5 to 10 bar, in particular from 0.75 to 5 bar. The reaction time can vary in dependence on various parameters, such as, for example, temperature, pressure, nature of the compound to be reacted, and can be determined for the process in question by the person skilled in the art using preliminary tests.

The particular compound of formula (B-II) obtained in step (b-I) can be isolated and/or purified by conventional methods known to persons skilled in the art.

Step (b-II)

Preferably, step (b-II) is a radical substitution reaction. Preferably, in step (b-II) the conversion of a compound according to formula (B-II) into a compound according to formula (I) is performed in the presence of at least one radical forming agent. Any radical forming agent suitable for the conversion of a compound according to formula (B-II) into a compound according to formula (I) may be employed.

Preferably, the radical forming agent is selected from the group consisting of tributyl tin hydride, tributyl tin anhydride, THF, phosphonic acid and trialkyl boranes. Most preferred radical forming agents are tributyl tin hydride and tributyl tin anhydride.

Preferably, in step (b-II) the radical forming agent is first decomposed by a radical starter such as azobisisobutyronitrile (AIBN) to form a radical. In case tributyl tin hydride is employed as radical forming agent, a tributyl tin radical is formed, which the reacts with the compound according to formula (B-II) to obtain a compound according to formula (I).

Preferably, in step (b-II) the compound according to formula (B-II) is dissolved or suspended in a suitable reaction medium together with the radical forming agent as well as the radical starter.

Suitable reaction media for the compound according to formula (B-II) are preferably selected from the group consisting of toluene, benzene, diethyl ether 1,4-dioxane, tetrahydrofuran (THF) and any mixtures in any ratio thereof. A particularly preferred reaction medium is toluene.

The reaction parameters for the reduction in step (b-II) such as, for example, pressure, temperature or reaction time, can independently of another vary over a wide range both. Preferably, the temperature in step (b-II) is in each case from 0 to 250° C., particularly preferably from 15 to 180° C. and very particularly preferably from 15 to 80° C. The reduction in step (b-II) can preferably be carried out at reduced pressure, at normal pressure or at elevated pressure, preferably in the range from 0.5 to 300 bar. It is particularly preferred to carry out the reactions under pressure in a range from 0.5 to 10 bar, in particular from 0.75 to 5 bar. The reaction time can vary in dependence on various parameters, such as, for example, temperature, pressure, nature of the compound to be reacted, and can be determined for the process in question by the person skilled in the art using preliminary tests.

The particular compound of formula (B-I) obtained in step (b-II) can be isolated and/or purified by conventional methods known to persons skilled in the art.

Step (b-III)

Optionally, the compound according to formula (I) may be converted into a physiologically acceptable acid addition salt thereof in step (b-III).

The conversion of a compound according to formula (I) into a corresponding acid addition salt may be carried out has it has been previously described in step (a-III) according to alternative A of the process of the invention.

Step (b-IV)

In a particularly preferred embodiment of the process of the invention, alternative B further comprises a deprotection step (b-IV), wherein the compound according to formula (I) or (B-II), wherein R¹ and R² have in each case have one of the above defined meanings and R³ in each case is ≠H, is deprotected to obtain a compound according to formula (Ib). The deprotection is preferably performed as it has been previously described for step (a-VI).

The deprotected compound according to formula (Ib) can be isolated and/or purified by conventional methods known to persons skilled in the art.

Optionally, the compound according to formula (Ib) may be converted into a physiologically acceptable acid addition salt thereof according to the procedure previously described for step (a-III).

### Process According to Alternative C

The process of the invention according to alternative C comprises at least the step (c-I), i.e. hydrogenation of a compound according to formula (C-I) to a compound according to formula (C-II) (step c-I), wherein R³ in each case has one of the above defined meanings, and optionally converting the thus obtained compound of formula (C-II) into a physiologically acceptable acid addition salt thereof (step c-II) or optionally converting the thus obtained compound of formula (C-II) into a compound according to formula (I), and optionally converting the thus obtained compound according to formula (I) into a physiologically acceptable acid addition salt thereof. Steps (c-I) and (c-III) are depicted in the following Scheme C1:

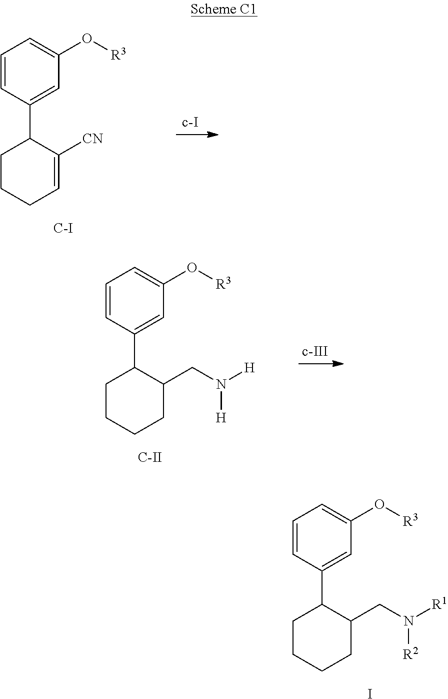

Step (c-I)

Preferably, the hydrogenation step (c-I) of the process of the invention according to alternative C is effected via heterogeneous or homogeneous catalysis, in each case in the presence of hydrogen. The hydrogen employed is preferably in gaseous form or at least part of it is dissolved in a liquid phase. In particular, the hydrogenation step (c-I) of the process of the invention according to alternative A is effected via heterogeneous catalysis.

If a homogeneous catalyst in hydrogenation step (c-I) according to alternative C of the process of the invention is employed, the same homogeneous catalysts as well as the same reaction parameters may be applied which may be also used for the hydrogenation reaction of step (a-I) of alternative A of the process of the invention.

If a heterogeneous catalyst in hydrogenation step (c-I) according to alternative C of the process of the invention is employed, the same heterogeneous catalysts as well as the same reaction parameters may be applied which may be also used for the hydrogenation reaction of step (a-I) of alternative A of the process of the invention.

In particular, a heterogeneous catalyst is employed in hydrogenation reaction step (c-I) in alternative C of the process of the invention. Preferred heterogeneous catalysts employed in this step are independently of another selected from the group consisting of Raney nickel, palladium, palladium on carbon (1-10 wt. %, preferably 5 wt. %), platinum, platinum on carbon (1-10 wt. %, preferably 5 wt. %), ruthenium on carbon (1-10 wt. %, preferably 5 wt. %) and rhodium on carbon (1-10 wt. %, preferably 5 wt. %). Most preferred is Raney Nickel as the catalyst for hydrogenation in step (c-I).

The compound according to formula (C-I) according to the process of the invention is preferably in liquid phase and to that end are preferably mixed with or dissolved in a reaction medium that is liquid under the particular reaction conditions. Examples of suitable reaction media employed in hydrogenation reactions are methanol, ethanol, isopropanol, n-butanol, n-propanol, toluene, n-heptane, n-hexane, n-pentane, acetic acid, ethyl acetate, formic acid, hydrochloric acid, hydrobromic acid, sulfuric acid and mixtures thereof. More preferably methanol is used as the reaction medium in step (c-I). Of course, it is also possible to use mixtures or multiphase systems comprising two or more of the above-mentioned liquids in the processes according to the present invention.

The reaction parameters for the hydrogenation reaction via heterogeneous catalysis in step (c-I) such as, for example, pressure, temperature or reaction time, can independently of another vary over a wide range both. Preferably, the temperature during the heterogeneous hydrogenation in step (c-I) is in each case from 0 to 250° C., particularly preferably from 15 to 180° C. and very particularly preferably from 15 to 30° C. The heterogeneous hydrogenation in step (c-I) can preferably be carried out at reduced pressure, at normal pressure or at elevated pressure, preferably in the range from 0.5 to 300 bar. It is particularly preferred to carry out the reactions under pressure in a range from 0.5 to 10 bar, in particular from 0.75 to 10 bar. The reaction time can vary in dependence on various parameters, such as, for example, temperature, pressure, nature of the compound to be reacted or the properties of the catalyst, and can be determined for the process in question by persons skilled in the art using preliminary tests.

The continuous removal of samples in order to monitor the reaction, for example by means of gas chromatography (GC) methods, is also possible, optionally in combination with regulation of the corresponding process parameters.

The total amount of the heterogeneous catalyst(s) used depends on various factors, such as, for example, the ratio of the catalytically active component to any inert material present, or the nature of the surface of the catalyst(s). The optimal amount of catalyst(s) for a particular reaction can be determined by persons skilled in the art using preliminary tests.

The particular compound of formula (C-II) obtained in step (c-I) can be isolated and/or purified by conventional methods known to persons skilled in the art.

Step (c-II)

The compound according to formula (C-II) obtained in step (c-I) corresponds to a compound according to formula (I), wherein R¹ and R² both denote H.

Optionally, the compound according to formula (C-II) may be converted into a physiologically acceptable acid addition salt thereof in step (c-II). The conversion of a compound according to formula (C-II) into a corresponding acid addition salt may be carried out has it has been previously described for the conversion of a compound according to formula (I) into a physiologically acceptable acid addition salt thereof in step (a-III) according to alternative A of the process of the invention.

Step (c-III)

Optionally, the compound according to formula (C-II) may be converted into a compound according to formula (I) in step (c-III), i.e. in a compound according to formula (I), wherein at least one of R¹ and R² denotes a C₁₋₄-aliphatic residue.

Any method suitable for substituting at least one hydrogen atom of a primary amine group with a C₁₋₄-aliphatic residue may be performed in step (c-III).

In a preferred embodiment, a compound according to formula (C-II) may be subjected to a reaction with a compound halogen substituted C₁₋₄-aliphatic compound, preferably a halogen substituted C₁₋₄-alkyl compound (C₁₋₄₋alkyl-Hal), wherein Hal in each case is preferably selected from the group consisting of Cl, Br and I to obtain a compound according to formula (I), wherein at least one of R¹ and R² denotes a C₁₋₄-aliphatic residue.

In another preferred embodiment, a compound according to formula (C-II) may be subjected to an Eschweiler-Clarke reaction in step (c-III). Preferably, a compound according to formula (C-II) is reacted with formaldehyde or a formaldehyde source such as paraformaldehyde, thereby generating a corresponding imine compound which is then further reacted with an acid, preferably an organic acid, more preferably, formic acid, thereby generating a compound according to formula (I), wherein at least one of R¹ and R² denotes a C₁₋₄-aliphatic residue, preferably both R¹ and R² denote a C₁₋₄-aliphatic residue, even more preferably both R¹ and R² denote a methyl group. Alternatively, a compound according to formula (C-II) is reacted with formaldehyde or a formaldehyde source such as paraformaldehyde, thereby generating a corresponding imine compound which is then further reacted with an hydrogen in combination with a catalyst, thereby generating a compound according to formula (I), wherein at least one of R¹ and R² denotes a C₁₋₄-aliphatic residue, preferably both R¹ and R² denote a C₁₋₄-aliphatic residue, even more preferably both R¹ and R² denote a methyl group.

Optionally, the thus obtained compound according to formula (I) may then be converted into a physiologically acceptable acid addition salt thereof, as has it has been previously described in step (a-III) according to alternative A of the process of the invention.

Steps (c-IV) and (c-V)

In a preferred embodiment of the process of the invention, alternative C further comprises a step (c-IV), wherein a compound of formula (C-0-I) is subjected to a desilylation reaction, wherein R³ is selected from the group consisting of H and a C₁₋₄-aliphatic residue, and wherein R^{a}, R^{b} and R^{c} are independently selected from the group consisting of C₁₋₈-aliphatic residues and aryl, preferably independently of another denote a C₁₋₈-aliphatic residue, even more preferably each denote methyl,

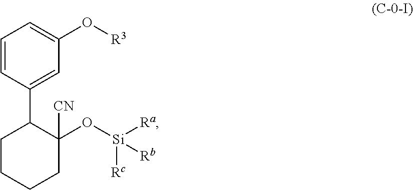

yielding a compound according to formula (C-0-II), wherein R³ has the above defined meaning,

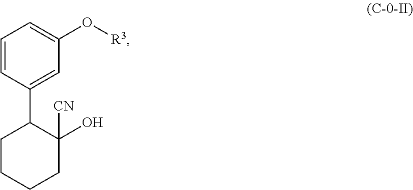

and step (c-V), wherein a compound of formula (C-0-II) is subjected to a dehydration reaction yielding the compound according to formula (C-I).

Steps (c-IV) and (c-V) are depicted in the following Scheme C2:

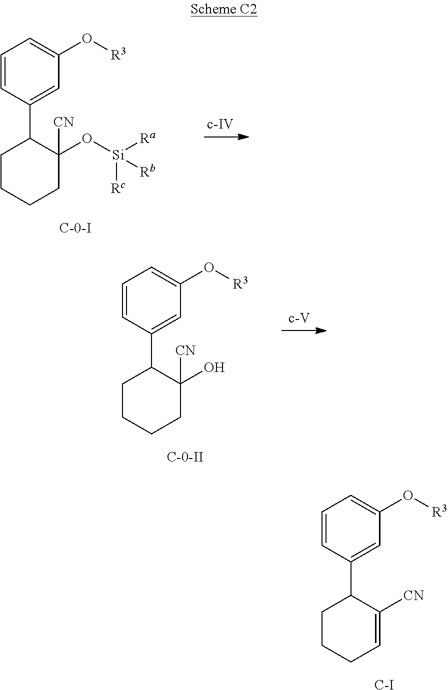

In a preferred embodiment of the present invention, the compound according to formula (C-0-11) is not isolated, i.e. a compound according to formula (C-0-I) can be directly transformed into a compound according to formula (C-I) in one step, i.e. steps (c-IV) and (c-V) can be carried out in one step (c-IV-V).

The desilylation step (c-IV) is preferably acid-catalyzed or acid-promoted or performed in the presence of a fluoride source such as potassium fluoride or cesium fluoride or tributylammonium fluoride. Alternatively, in case R^{a}, R^{b} and R^{c} each denote methyl, (c-IV) may also be performed in the presence of a base, preferably an inorganic base such as potassium carbonate. However, in a most preferred embodiment desilylation step (c-IV) is performed in the presence of an acid in a catalytically effective or at least stoichiometric amount. Preferably the acid is selected from the group consisting of formic acid, hydrochloric acid, acetic acid, sulfuric acid, hydrobromic acid, methanesulfonic acid, phosphoric acid or any mixture thereof. It is preferable if the acid is employed in a high concentration. Particularly preferably, hydrochloric acid is employed.

Examples of suitable reaction media for (c-IV) include lower alcohols such as methanol or ethanol as well as THF, 1,4-dioxane or any mixture thereof. Of course, it is also possible to use mixtures or multiphase systems comprising two or more of the above-mentioned liquids in the processes according to the present invention.

The particular compound of general formula (C-0-11) obtained can be isolated and/or purified by conventional methods known to persons skilled in the art.

The dehydration step (c-V) is preferably acid-catalyzed or acid-promoted, i.e. performed in the presence of an acid in a catalytically effective or at least stoichiometric amount. Preferably the acid is selected from the group consisting of formic acid, hydrochloric acid, acetic acid, sulfuric acid, hydrobromic acid, methanesulfonic acid, p-toluenesulfonic acid, phosphorous pentoxide, thionyl chloride, phosphoryl chloride or any mixture thereof. It is preferable if the acid is employed in a high concentration. Particularly preferably, phosphoryl chloride employed.

The compound of formula (C-0-11) used in step (c-V) according to the present invention is preferably in liquid phase and to that end is preferably mixed with or dissolved in a reaction medium that is liquid under the particular reaction conditions. Examples of suitable reaction media include acetic acid, formic acid, toluene, pyridine, hydrochloric acid, sulfuric acid, hydrobromic acid, methanesulfonic acid, p-toluenesulfonic acid, phosphorous pentoxide, thionyl chloride, phosphoryl chloride or any mixture thereof. Of course, it is also possible to use mixtures or multiphase systems comprising two or more of the above-mentioned liquids in the processes according to the present invention.

Preferably, step (c-V) is performed in pyridine as a reaction medium in the presence of phosphoryl chloride.

The reaction parameters for step (c-V), such as, for example, pressure, temperature or reaction time, can vary over a wide range. It is preferable if the reaction temperature in step (c-V) is between 15 and 100° C., particularly preferably between 18 and 90° C. The dehydration step (c-V) can preferably be carried out at reduced pressure, at normal pressure or at elevated pressure, preferably in the range from 0.01 to 300 bar. It is particularly preferred to carry out the reactions under pressure in a range from 0.5 to 5 bar, in particular from 0.5 to 1.5 bar.

The reaction time can vary in dependence on various parameters, such as, for example, temperature, pressure, nature of the compound to be reacted or the properties of the catalyst, and can be determined for the process in question by persons skilled in the art using preliminary tests. It is preferable if the reaction time of step (c-V) is between 2 and 25 hours, particularly preferably between 3 and 22 hours, more particularly preferably between 4 and 20 hours.

The continuous removal of samples in order to monitor the reaction, for example by means of gas chromatographic (GC) methods, is also possible, optionally in combination with regulation of the corresponding process parameters.

The particular compound of general formula (C-I) obtained can be isolated and/or purified by conventional methods known to persons skilled in the art.

Step (c-VI)

In a particularly preferred embodiment of the process of the invention, alternative C further comprises a deprotection step (c-VI), wherein one of the compounds according to formula (C-I), (C-II), (C-0-I), (C-0-II) or (I), wherein R¹ and R² have in each case have one of the above defined meanings and R³ in each case is ≠H, is deprotected to obtain a compound according to formula (Ib).

Preferably, the deprotection step (c-VI) is carried out by subjecting a compound according to formula (I), (C-I), or (C-II), more preferably a compound according to formula (C-I), to said deprotection.

Preferably, at least one acid, preferably at least one acid selected from the group consisting of hydrobromic acid, hydrochloric acid and methanesulfonic acid is employed as deprotecting agent in step (c-VI). In case methanesulfonic acid is employed as acid a combination of methanesulfonic acid and methionine is preferably used as as deprotecting agent. A combination of methanesulfonic acid and methionine is the most preferred deprotecting agent in step (c-VI). The deprotection step (c-VI) is preferably carried out in a reaction medium selected from the group consisting of diethylether, tetrahydrofuran, toluene, 2-methyltetrahydrofuran, dioxane, tert.-butyl methylether and any mixture thereof.

The reaction parameters for step (c-VI), such as, for example, pressure, temperature or reaction time, can vary over a wide range. It is preferable if the reaction temperature in step (c-VI) is between 15 and 100° C., particularly preferably between 18 and 80° C. Preferably, step (c-VI) is carried out at normal pressure.

The reaction time can vary in dependence on various parameters, such as, for example, temperature, pressure, nature of the compound to be reacted and can be determined for the process in question by the person skilled in the art using preliminary tests. It is preferable if the reaction time of step (c-VI) is between 2 and 25 hours, particularly preferably between 3 and 22 hours, more particularly preferably between 4 and 20 hours.

The particular deprotected compound of can be isolated and/or purified by conventional methods known to persons skilled in the art.

Step (c-VII)

In a preferred embodiment of the process of the invention, alternative C further comprises a step (c-VII) for the preparation of a compound according to formula (C-0-I) as depicted in the following Scheme C4:

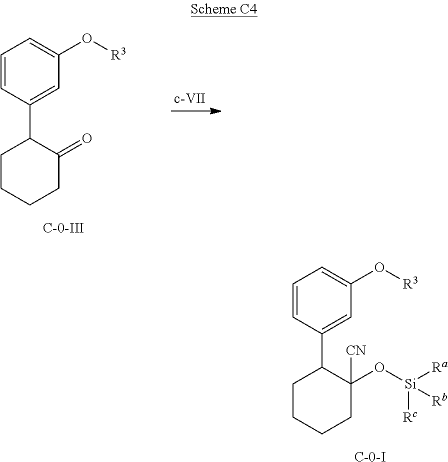

In step (c-VII) a compound according to formula (C-0-III), wherein R³ has one of the above defined meanings, is reacted with a compound having the formula SiR^{a}R^{b}R^{c}(CN), wherein R^{a}, R^{b} and R^{c} are independently of another selected from the group consisting of C₁₋₈-aliphatic residues and aryl, preferably independently of another denote a C₁₋₈-aliphatic residue. Preferably the compound SiR^{a}R^{b}R^{c}(CN) is selected from the group consisting of trimethylsilylcyanide, triethylsilylcyanide, tri-n-propylsilylcyanide and triisopropylsilylcyanide. Most preferred is trimethylsilylcyanide.

A suitable reaction medium for step (c-VII) is preferably at least one reaction medium selected from the group consisting of acetone, benzene, n-butanol, 2-butanone, tert.-butyl methylether, chloroform, cyclohexane, diethyl ether, 1,4-dioxane, diisopropyl ether, alkyl acetates, e.g. ethyl acetate, ethanol, n-hexane, n-heptane, isopropanol, methanol, methylene chloride (dichloromethane), n-pentane, petrol ether, n-propanol, tetrahydrofuran, toluene and any mixture in any mixing ratio thereof. Most preferred reaction media are n-hexane and n-heptane.

Preferably, step (c-VII) of the process of the invention according to alternative C is performed in the presence of at least metal halide, preferably at least one transition metal halide, wherein the halide is preferably selected from the group consisting of chloride, bromide and iodide. Most preferred is a zinc halide, in particular zinc iodide (ZnI₂).

The thus obtained compound according to formula (C-0-I) of can be isolated and/or purified by conventional methods known to persons skilled in the art.

### Process According to Alternative D

The process of the invention according to alternative D comprises at least the step (d-I), i.e. reacting a magnesium halide formed from a compound according to formula (D-I), wherein Hal is a halogen atom and wherein R³ has the above defined meaning, preferably wherein R³ denotes a C₁₋₄-aliphatic residue, with a compound according to formula (D-II), wherein Hal is a halogen atom and wherein R¹ and R² have the above defined meanings,

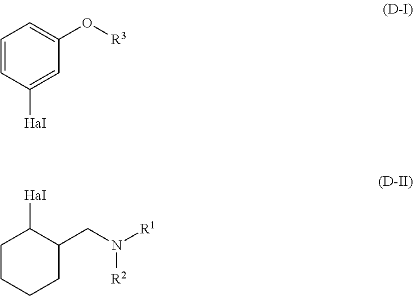

to a compound according to formula (I), wherein R¹, R² and R³ have the above defined meanings, preferably wherein R¹ and R² have the above defined meanings and R³ is a C₁₋₄-aliphatic residue, and optionally a step (d-II), wherein the compound according to formula (I) is converted into a physiologically acceptable acid addition salt thereof.

In step (d-I) a magnesium halide, i.e. a Grignard reagent is formed from a compound according to formula (D-I), wherein Hal is a halogen atom, preferably selected from the group consisting of Cl, Br and I, in particular Br, and magnesium in an inert reaction medium. Said Grignard reagent is then reacted with a compound according to formula (D-II), preferably in an inert reaction medium, more preferably in an organic ether, for example, selected from the group consisting of diethyl ether, tetrahydrofuran, 2-methyltetrahydrofuran, tert-butylmethyl ether, dioxan or any mixture thereof to obtain a compound according to formula (I).

The reaction of the compound according to formula (D-II) with the Grignard reagent formed from a compound according to formula (D-I) and magnesium is preferably effected via transition metal cataylsis, more preferably effected via transition metal cataylsis, wherein the transition metal is selected from the group consisting of Cu, Ag, Au, Zn, Cd, Hg, V, Nb, Ta, Cr, Mo, W, Fe, Ru, Os, Co, Rh, Ir, Ni, Pd and Pt, preferably from the group consisting of Cu, Ag, Zn, Fe and Mn, more preferably from the group consisting of Cu and Fe, and in particular Fe, even more preferably catalzyed by a transition metal complex, still more preferably catalzyed by a transition metal complex, wherein the transition metal is selected from the group consisting of Cu, Ag, Au, Zn, Cd, Hg, V, Nb, Ta, Cr, Mo, W, Fe, Ru, Os, Co, Rh, Ir, Ni, Pd and Pt, preferably from the group consisting of Cu, Ag, Zn, Fe and Mn, more preferably from the group consisting of Cu and Fe, and in particular Fe. Such a transition metal complex can be formed from a transition metal, which may have different oxidation states, e.g. in case of an iron complex such a complex may contain iron(0) and/or iron(II) and/or iron(III) and suitable ligands, such as e.g. diphoshine ligands.

The particular compound of formula (I) obtained can be isolated and/or purified by conventional methods known to persons skilled in the art.

Any stereoisomers of any compounds obtained via alternatives A, B, C or D of the process of the invention, such as e.g. a compound according to any of the formulae (I-1), (I-2), (I-3), (I-4), (I-1a), (I-2a), (I-3a), (I-4a), (PI-1), (PI-2), (PI-3) and (PI-4) may be separated from each other at any time of each reaction sequence and/or purified by conventional methods known to a person skilled in the art. Preferably, LC (liquid chromotography) techniques, more preferably HPLC (high performance liquid chromatography) techniques, even more preferably semi-preparative or preparative HPLC techniques may be used for the separation of these stereoisomers. Alternatively, fractionized crystallization of any mixture of these stereoisomers may be carried out to separate any unwanted stereoisomers.

The steps according to each of the three alternatives according to the present invention can be carried out discontinuously (batchwise) or continuously, preference being given to the discontinuous procedure.

There come into consideration as the reactor for the discontinuous procedure, for example, a slurry reactor, and for the continuous procedure a fixed-bed reactor or loop reactor.

The present invention is illustrated in further detail hereinafter by a number of examples. The illustrations are only illustrative and do not limit the present invention to the examples.

### EXAMPLES

### Abbreviations

AIBN: azo-bis-isobutyronitrile
AlCl₃: aluminium chloride
Eq.: equivalents
CC: column chromatography on silica gel
DCC: N,N′-dicyclohexyl carbodiimide
THF: tetrahydrofuran
TEA: triethylamine
h: hour(s)
HPLC: high performance liquid chromatography
HBr: hydrobromic acid
HCl: hydrochloric acid
MgSO₄: magnesium sulfate
NH₄HSO₄: ammonium hydrogen sulfate
NaOH: sodium hydroxide
NaCl: sodium chloride
NaHCO₃: sodium hydrogen carbonate
Na₂CO₃: sodium carbonate
KOtBu: potassium tent-butylate
LAH: lithium aluminium hydride
HOBT (1-HBT): 1-hydroxybenzotriazol
RT: room temperature
TMSCN: trimethylsilylcyanide
TBAB: tetra-n-butylammoniumbromide
GC-MS: gas chromatographic/mass spectrometric analysis
“M” and “N” are concentrations in mol/l. “%” are volume-% if not indicated otherwise.

The yields of any compounds obtained in any steps of the process of the invention have not been optimized. Any temperatures were not corrected.

All compounds not explicitly described were either commercially available (e.g. from Acros, Avocado, Aldrich, Bachem, Fluka, Lancaster, Maybridge, Merck, Sigma, TCI, Oakwood etc.) (syntheses of these compounds may e.g. be researched in the “Symyx® Available Chemicals Database” of the company MDL, San Ramon, US) or the syntheses of these compounds has been already described by technical literature sources (experimental procedures may e.g. by researched in the “Reaxys®” database of the company Elesevier, Amsterdam, NL) or these compounds may be synthesized according to conventional procedures known to a person skilled in the art.

Silica gel 60 (0.010-0.063 mm; company: Merck, Darmstadt, Germany) was used as stationary phase for CC (column chromatography). Mixing ratios of any solvent or eluent mixtures are indicated in volume/volume.

The analytical characterization of all compounds was performed by means of ¹H-NMR spectroscopy and mass spectrometric analyses.

A process according to alternative A for preparing different stereoisomers of (3-(2-(dimethylamino)methyl)cyclohexyl)phenol is shown and described in the following Scheme A:

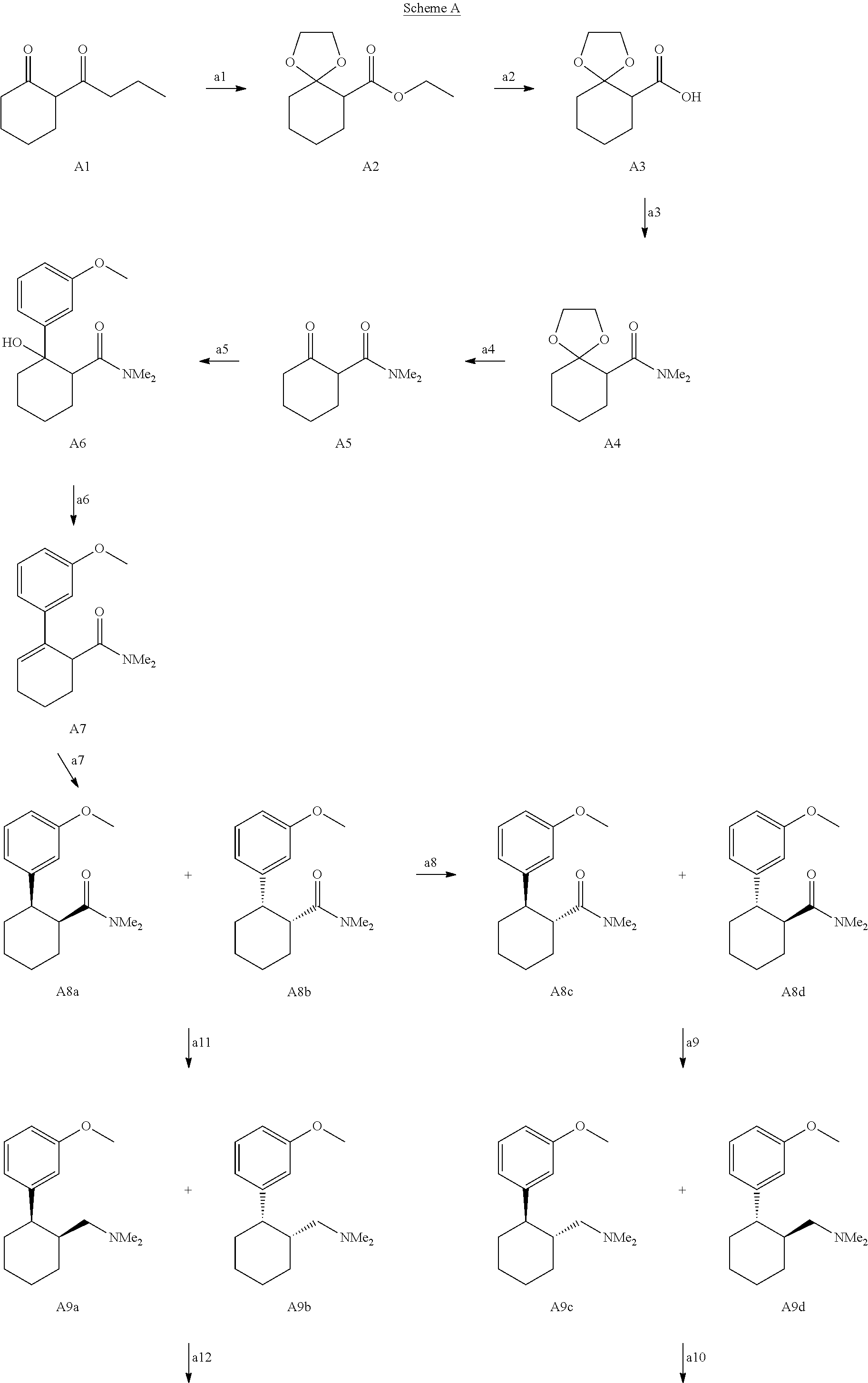

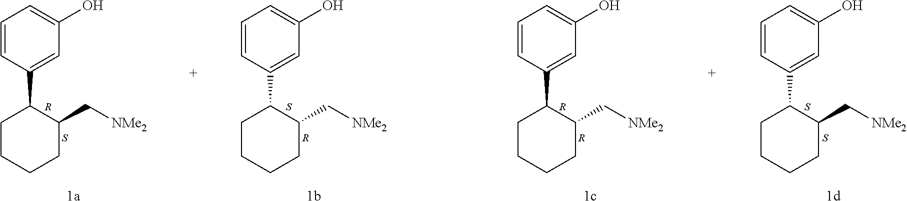

### Step a1: Compound A2 (1,4-dioxa-spiro[4.5]decan-6-carbonic acid ethyl ester)

80 ml (0.5 mol) A1 and 34.2 g (1.1 mol) ethylene glycol, 1.0 g p-toluene sulfonic acid were dissolved in 380 mL toluene and refluxed in a Dean-Stark apparatus for 24 h. The organic layer was washed with a saturated aqueous solution of NaHCO₃ and dried over MgSO₄. After removal of the organic solvent under reduced pressure, the residue was dried in vacuo. 103.2 g (96%) A2 were obtained in form of a colorless oil.

### Step a2: Compound A3 (1,4-dioxa-spiro[4.5]decan-6-carbonic acid)

To 103.2 g (0.482 mol) of A2 were added 77 g of NaOH (1.93 mol), 200 mL water and 1 L of THF and the resulting mixture was refluxed for 12 h. Acetic acid was added until the solution had a pH value of 4. The organic layer was washed with water. After addition of a saturated aqueous solution of NaCl, the organic layer was separated and dried over MgSO₄. After removal of the organic solvent, the residue was dissolved in a small amount of ethyl acetate. A layer of 700 mL of n-hexane was slowly added and the resulting solution was cooled at −20° C. for 72 h. The resulting crystals of A3 were filtered off and dried in vacuo (yield: 36 g (40%)).

### Step a3: Compound A4 (1,4-dioxa-spiro[4.5]decan-6-carbonic acid dimethyl amide)

9.0 g (48.4 mmol) A3, 4.7 g (58.1 mmol) dimethylamine hydrochloride, 12 g (58.1) mmol DCC, 7.8 g (58.1 mmol) HOBT and 13.5 ml (96.8 mmol) TEA were added to 120 mL of dichloromethane and stirred for 48 h at room temperature. The resulting colorless solid (dicyclohexyl urea) was filtered off and removed. To the filtrate 100 mL of ethyl acetate were added. After washing with an aqueous solution of citric acid (5%), an aqueous solution of NaOH (2N) and a saturated aqueous solution of NaCl, the organic layer was separated and dried over MgSO₄. The organic solvent was evaporated under reduced pressure and the remaining residue was dried in vacuo. 10.1 g (99%) of A4 were obtained in form of a colorless solid.

### Step a4: Compound A5 (N,N-dimethyl-2-oxocyclohexanecarboxamide)

10.5 g (0.05 mol) A4 were dissolved in 140 mL of THF and cooled to +4° C. 80 mL of an aqueous solution of NaCl (32%) were added and the resulting solution was stirred for 2 h at +4° C. An aqueous solution of NaOH was added to this solution at +4° C. until the solution had a pH value of 7-8. The organic layer was separated and dried over MgSO₄. The organic solvent was evaporated under reduced pressure and the remaining residue was dried in vacuo. CC (eluent: n-hexane/ethyl acetate/methanol (16:4:2)) yielded 4.3 g (51%) of A5 as a colorless solid.

### Step a5: Compound A6 (2-hydroxy-2-(3-methoxyphenyl)-N,N-dimethylcyclohexanecarboxamide)

To 0.73 g (29.9 mmol) of magnesium were added 50 mL of dry THF and the resulting suspension was refluxed. A solution of 3.79 mL of 3-bromoanisole in 10 mL of dry THF was slowly added dropwise. After addition, the remaining suspension was refluxed for another 2 h and then cooled to +4° C. At this temperature, 3.36 g (19.9 mmol) of A5, dissolved in 20 mL of dry THF were slowly added dropwise. After stirring for 18 h at room temperature, the reaction mixture was hydrolyzed by addition of 100 mL of a saturated aqueous solution of NH₄HSO₄. The organic layer was separated and dried over MgSO₄. The organic solvent was evaporated under reduced pressure and the remaining residue was dried in vacuo. CC (eluent: n-hexane/ethyl acetate (2:1)) yielded 2.2 g (43%) of A6 (melting point 41.4° C.) as a colorless crystalline solid.

### Step a6: Compound A7 (2-(3-methoxyphenyl)-N,N-dimethylcyclohex-2-enecarboxamide)

2.9 g (3.24 mmol) A6 were dissolved in 35 mL of an aqueous solution of HBr (47%) and heated to 50° C. for 7 h. The reaction mixture was then cooled to RT. 50 mL of ethyl acetate were added and the resulting mixture was neutralized with solid NaHCO₃. The organic layer was separated and the aqueous layer was extracted with ethyl acetate several times. The combined organic layers were dried over MgSO₄. The organic solvent was evaporated under reduced pressure and the remaining residue was dried in vacuo. 2.36 g (87%) of A7 was obtained in form of a colorless solid.

### Step a7: Compounds A8a and A8b ((1RS,2SR)-2-(3-methoxyphenyl)-N,N-dimethylcyclohexanecarboxamide)

2.3 g (8.9 mmol) A7 were placed in a hydrogenation apparatus and dissolved in 50 mL of methanol. 3 drops of an aqueous solution of HCl (32%) and 300 mg Pd/C (10% Pd) as catalyst were added under inert gas atmosphere. The resulting mixture was hydrogenated at room temperature under a H₂-pressure of 1 bar (500 mL H₂-reservoir) for 12 h. The solids were filtered off and washed with methanol. The methanol portion and the filtrate were combined. After evaporation of the organic solvents, the residue was suspended in a mixture of water and diethyl ether. A diluted aqueous solution of NaOH was added to the mixture. The layers of the resulting solution were separated and the aqueous layer was washed three times with diethyl ether. The combined organic layers were dried over MgSO₄. The organic solvent was evaporated under reduced pressure and the remaining residue was dried in vacuo. 2.04 g (88%) of A8a and A8b were obtained in form of a slightly purple colored oil.

### Step a8: Compounds A8c and A8d ((1RS,2RS)-2-(3-methoxyphenyl)-N,N-dimethylcyclohexanecarboxamide)

To 412 mg (1.58 mmol) of A8a and A8b were added 25 mL of dry THF and 1.5 g (13.3 mmol) KOtBu. The resulting mixture was refluxed for 12 h. After cooling to RT diethyl ether was added and the mixture was hydrolyzed at +4° C. with a diluted aqueous solution of HCl. NaHCO₃ was added to this solution at +4° C. until the solution had a pH value of 6-7. The layers of the resulting solution were separated and the aqueous layer was extracted several times with diethyl ether. The combined organic layers were dried over MgSO₄. The organic solvent was evaporated under reduced pressure and the remaining residue was dried in vacuo. 400 mg (97%) of A8c and A8d were obtained in form of a colorless oil.

### Step a9: Compounds A9c and A9d ((1RS,2RS)-2-(3-methoxyphenyl)cyclohexyl)-N,N-dimethylmethanamine)

To a mixture of 203 mg (5.35 mmol) LAH and 179 mg (1.34 mmol) AlCl₃ in 25 mL dry THF 280 mg (1.07 mmol) of A8c and A8d were added at RT. The resulting mixture was then refluxed for 6 h. After addition of 25 mL of a saturated aqueous solution of NH₄HSO₄, Na₂CO₃ was added until the solution had a pH value of about 8. The layers were separated from each other and the aqueous layer was extracted five times with diethyl ether. The combined organic layers were dried over MgSO₄. The organic solvent was evaporated under reduced pressure and the remaining residue was dried in vacuo. 230 mg (91%) of A9c and A9d were obtained in form of a colorless oil. The hydrochloride salts of A9c and A9d were obtained by addition of trimethylsilylchloride (1.2 eq.) at 0° C. to a solution of A9c and A9d in acetone (yield: 190 mg (75%) in form of a colorless solid)).

### Step a10: Compounds 1c and 1d ((1RS,2RS)-3-(2-dimethylaminomethyl-cyclohexyl)-phenol)

To 150 mg (0.53 mmol) of the hydrochloride of A9c and A9d were added 4 mL of an aqueous solution of HBr (47%). The resulting mixture was stirred for 2 h at 100° C. After cooling to +4° C., NaHCO₃ was added until the solution had a pH value of about 6-7. The solution was extracted three times with diethyl ether. The combined organic layers were dried over MgSO₄. The organic solvent was evaporated under reduced pressure and the remaining residue was dried in vacuo. 90 mg (73%) of 1c and 1d were obtained in form of a colorless oil.

### Step a11: Compounds A9a and A9b ((1RS,2SR)-(2-(3-methoxyphenyl)cyclohexyl)-N,N-dimethylmethanamine

To a mixture of 1.3 mL (3.00 mmol) of LAH-THF solution (2.3 M) and 233 mg (1.75 mmol) AlCl₃ in 5 mL dried THF 1600 mg (0.60 mmol) of A8a and A8b were added at RT. The resulting mixture was then refluxed for 6 hours. After addition of 25 mL of a saturated aqueous solution of NH₄HSO₄, Na₂CO₃ was added until the solution had a pH value of about 8. The layers were separated from each other and the aqueous layer was extracted five times with diethyl ether. The combined organic layers were dried over MgSO₄. The organic solvent was evaporated under reduced pressure and the remaining residue was dried in vacuo. 148 mg (99%) of A9a and A9b were obtained in form of a colorless oil. The hydrochloride salt of A9a and A9b was obtained by addition of trimethylsilylchloride (1.2 eq.) to a solution of A9a and A9b in diethyl ether at 0° C. (yield: 120 mg (71%) in form of a colorless solid)).

### Step a12: Compounds 1a and 1b ((1RS,2SR)-3-(2-dimethylaminomethyl-cyclohexyl)-phenol)

To 400 mg (1.44 mmol) of the hydrochlorides of A9a and A9b were added 6 mL of an aqueous solution of HBr (47%). The resulting mixture was stirred for 2 h at 100° C. After cooling to +4° C., NaHCO₃ was added until the solution had a pH value of about 6-7. The solution was extracted three times with diethyl ether. The combined organic layers were dried over MgSO₄. The organic solvent was evaporated under reduced pressure and the remaining residue was dried in vacuo. 210 mg (64%) of 1a and 1b were obtained in form of a colorless oil. The hydrochloride salts of 1a and 1b were obtained by addition of trimethylsilylchloride (1.2 eq.) at 0° C. to a solution of 1a and 1b in isopropanol (yield: 70 mg (19%) in form of a colorless solid)).

A process according to alternative B for preparing ((1RS,2SR)-2-(3-methoxyphenyl)-cyclohexyl)-N,N-dimethylmethanamine is shown and described in the following Scheme B:

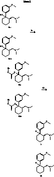

### Step b1: Compounds B2a and B2b (O-(1RS,2SR)-2-((dimethylamino)methyl)-1-(3-methoxyphenyl)cyclohexyl O-phenyl carbonothioate)

0.5 g (1.9 mmol) B1a and B1b were dissolved in 7.5 ml dry THF. The resulting solution was cooled to −70° C. via an acetone/nitrogen bath. 0.51 mL (3.8 mmol) phenylchlorothioformate were added to the mixture yielding a colorless precipitate. The reaction mixture was stirred for 45 minutes, allowed to warm to RT and then extracted with 40 mL diethyl ether. The combined organic layers including the precipitate were then extracted three times with a saturated aqueous ammonium chloride solution thereby leading to a dissolution of the precipitate. The combined organic layers were dried over MgSO₄. The solvents were evaporated under reduced pressure and the remaining residue was dried in vacuo. Recyrstallization from diethyl ether yielded 0.19 g (23%) of the hydrochloride salts of B2a and B2b in form of colorless crystals. The free bases B2a and B2b were obtained by addition of NaHCO₃ to a mixture of the hydrochlorides of B2a and B2b in water and dichloromethane.

### Step b2: Compounds 2a and 2b ((1RS,2SR)-2-(3-methoxyphenyl)cyclohexyl)-N,N-dimethylmethanamine)

150 mg (0.4 mmol) B2a and B2b were dissolved in 5 mL dry toluene under inert gas atmosphere. To this solution 0.33 mL (1.2 mmol) tributyl tinhydride and a small amount of AIBN (azo-bis-isobutyronitrile) covering the tip of a spatula were added. The resulting solution was stirred for 3 h at a temperature of 100° C. (temperature of the oil bath) and then stirred at RT for another 16 h. The solvents were evaporated under reduced pressure. 20 mL of an aqueous solution of HCl (17%) were added to the resulting residue. The resulting solution was then extracted with 20 mL of diethyl ether. The layers were separated and the organic layer was extracted twice with 20 mL of an aqueous solution of HCl. The combined aqueous layers were then washed several times with diethyl ether and then alkalized with NaOH and then extracted with a mixture of diethyl ether and ethyl acetate. The layers were separated and the organic layer was dried over MgSO₄. The solvent were evaporated under reduced pressure and the remaining residue was dried in vacuo. 0.09 g (91%) of 2a and 2b in form of a colorless oil was obtained.

A process according to alternative C for preparing 3-(1-amino-2-methylpentan-3-yl)phenol is shown and described in the following Scheme C:

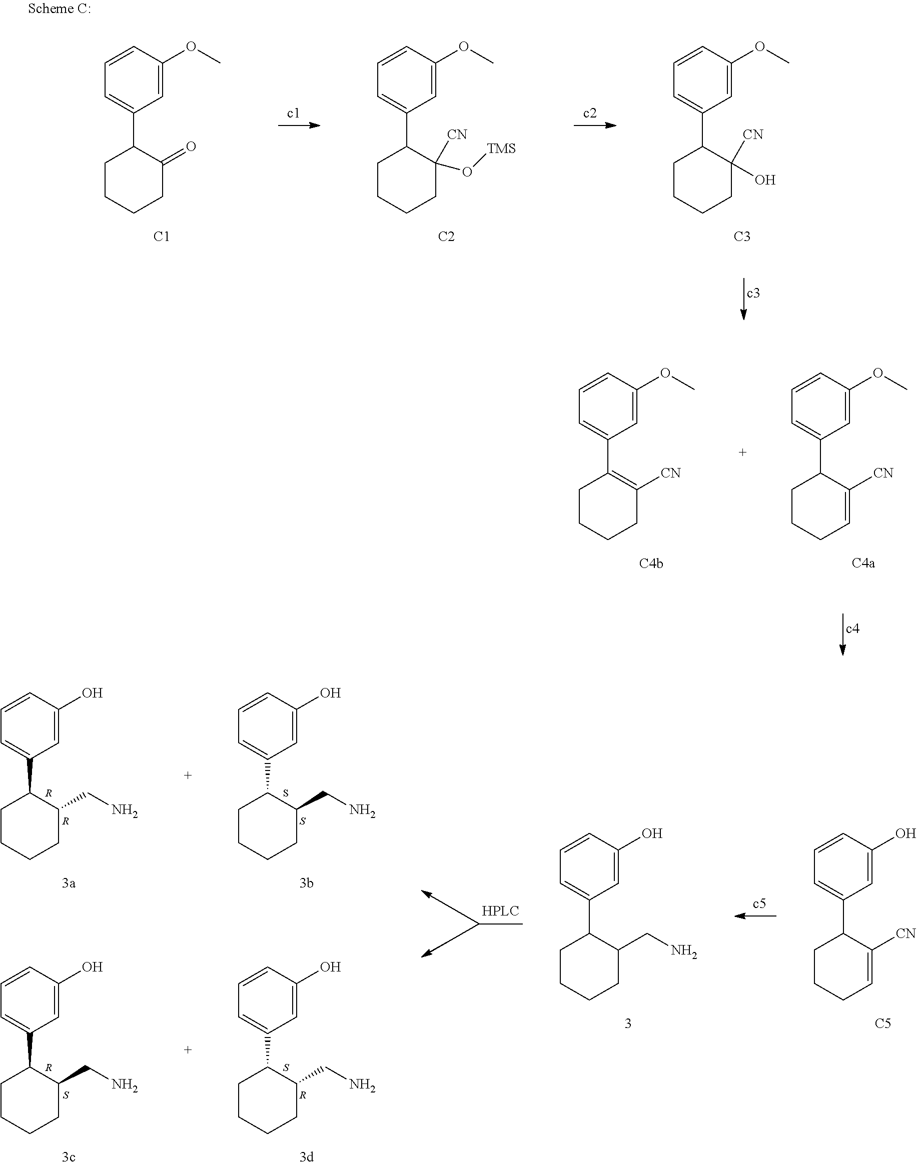

### Step c1: Compound C2 (2-(3-methoxy-phenyl)-1-trimethylsilanyloxy-cyclohexanecarbonitrile)

To 1.8 g Zn1₂ were added 13.9 g (68 mmol) of commercially available 2-(3-methoxyphenyl)-cyclohexanone (C1) (Acros) and 10 mL (75 mmol) TMSCN. The resulting mixture was stirred for 90 minutes. After addition of 70 mL of dry n-hexane, the mixture was refluxed for another 15 minutes. After addition of a small amount of charcoal covering the tip of a spatula, the resulting mixture was refluxed for another 15 minutes. The reaction mixture was then filtered and the residue was washed with 200 mL of n-hexane. The filtrate and the n-hexane washing solution were then combined and the solvents were evaporated under reduced pressure. 17.83 g (86%) of C2 were obtained in form of a brownish yellow oil.

### Step c2: Compound C3 (1-hydroxy-2-(3-methoxy-phenyl)-cyclohexancarbonitrile)

To 1.13 g (3.7 mmol) C2 were added 11 mL methanol and the resulting mixture was cooled to 0° C. 2.7 ml of an aqueous solution of HCl (5 M) were added at this temperature and the mixture was stirred for 4 h at RT. The organic solvents were evaporated under reduced pressure and a mixture of water and diethyl ether was added to the remaining residue. NaHCO₃ was added until the aqueous layer had been neutralized. The layers were separated and the aqueous layer was extracted several times with diethyl ether. The combined organic layers were dried over MgSO₄. The organic solvent was evaporated under reduced pressure and the remaining residue was dried in vacuo. 0.84 g (99%) of C3 were obtained in form of a brownish yellow oil.

### Step c3: Compounds C4a (6-(3-methoxy-phenyl)-cyclohex-1-ene-carbonitrile) and C4b (2-(3-methoxyphenyl)cyclohex-1-ene-carbonitrile)

To 1.07 g (4.6 mmol) C3 were added 28 mL toluene, 28 mL pyridine and 36 mL of phosphoryl chloride (POCl₃), dissolved in 16 mL pyridine. The resulting mixture was refluxed for 1 h. After cooling, the reaction mixture was poured into ice water and extracted with diethyl ether. The combined organic layers were dried over MgSO₄. The organic solvent was evaporated under reduced pressure and the remaining residue was dried in vacuo. CC (eluent: n-hexane/diethyl ether (10:1)) yielded 0.4 g (41%) of C4a well as 0.37 g (38%) of regioisomeric C4b, each as a yellowish oil.

### Step c4: Compound C5 (6-(3-hydroxy-phenyl)-cyclohex-1-ene-carbonitrile

To 0.4 g D,L-methionine and 5 mL methane sulfonic acid were added 0.39 g (1.83 mmol) C4a. The reaction mixture was stirred for 16 h. Water and ethyl acetate were added. After neutralization with solid NaHCO₃ the layers were separated from each other. The aqueous layer was extracted twice with ethyl acetate. The combined organic layers were dried over MgSO₄. The organic solvents were evaporated under reduced pressure and the remaining residue was dried in vacuo. CC (eluent: ethyl acetate/n-hexane (5:1)) yielded 0.13 g (37%) of C5 in form of a brownish oil.

### Step c5: Compound 3 (3-(2-aminomethyl-cyclohexyl)-phenol)

0.13 g (0.65 mmol) C5 were dissolved in 40 mL of dry methanol in a hydrogenation apparatus. A small amount of a commercially available Raney-Nickel (Ra—Ni) suspension in water covering the tip of a spatula was added to the mixture. The resulting mixture was hydrogenated at room temperature under a H₂-pressure of 2 bar for 12 h. The solids were filtered off and washed with dried methanol. The methanol portion and the filtrate were combined. The combined organic layers were dried over MgSO₄. Methanol was evaporated under reduced pressure and the remaining residue was dried in vacuo to obtain compound 3. HPLC (preparative scale, column: Hypercarb 5 μm, 250·21.2 mm, eluent methanol/water (85:15)+0.1% diethylamine) yielded 25 mg (19%) of 3a and 3b and 50 mg of 3c and 3d (37%), each in form of a colorless oil.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A process for preparing a compound corresponding to formula (I) 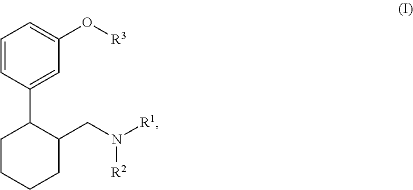
wherein
R¹, R² and R³ are each independently selected from the group consisting of H and C₁₋₄-aliphatic residues,
or a physiologically acceptable acid addition salt thereof,
according to alternative A comprising the steps of:
(a-I) hydrogenating a compound corresponding to formula (A-I): 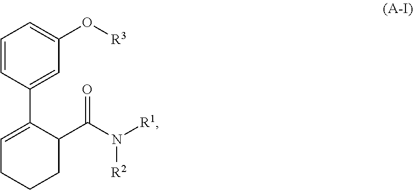
wherein R¹, R² and R³ have the above defined meanings,
to a compound corresponding to formula (A-II), 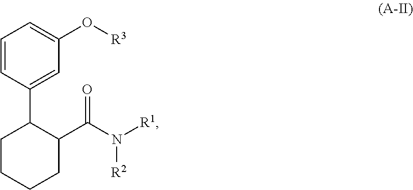
wherein R¹, R² and R³ have the above defined meanings,
(a-II) reducing the resulting compound corresponding to formula (A-II) to a compound corresponding to formula (I), and
(a-III) optionally converting the compound corresponding to formula (I) into a physiologically acceptable acid addition salt thereof,
or according to alternative B comprising the steps of:
(b-I) converting a compound corresponding to formula (B-I): 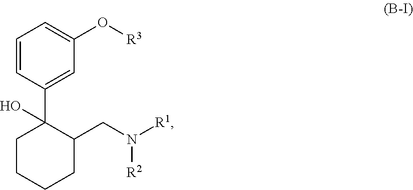
wherein R¹, R² and R³ have the above defined meanings,
into a compound corresponding to formula (B-II): 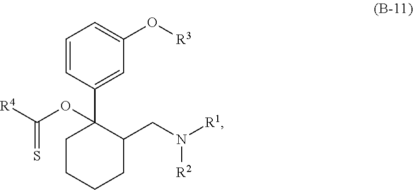
wherein
R¹, R² and R³ have the above defined meanings, and
R⁴ denotes (Y)ₙ—R⁵, wherein
n denotes 0 or 1,
Y denotes S, O, NH or an N(C₁₋₄-aliphatic residue), and
R⁵ is selected from the group consisting of C₁₋₈-aliphatic residues, C₃₋₈-cycloaliphatic residues, aryl, heteroaryl, C₁₋₄-alkylene-C₃₋₈-cycloaliphatic residues, C₁₋₄-alkylene-aryl and C₁₋₄-alkylene-heteroaryl,
(b-II) converting the resulting compound corresponding to formula (B-II) into a compound corresponding to formula (I), wherein R¹, R² and R³ have the above defined meanings, and
(b-III) optionally converting the compound corresponding to formula (I) into a physiologically acceptable acid addition salt thereof,
or according to alternative C comprising the steps of:
(c-I) hydrogenating a compound corresponding to formula (C-I): 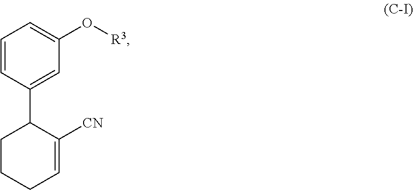
wherein R³ has the above defined meaning,
to a compound according to formula (C-II): 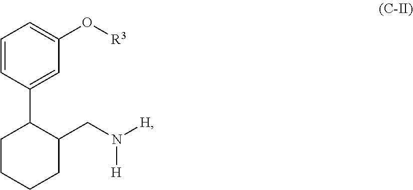
wherein R³ has the above defined meaning, and
(c-II) optionally converting the resulting compound of formula (C-II) into a physiologically acceptable acid addition salt thereof, or
(c-III) optionally converting the resulting compound of formula (C-II) into a compound corresponding to formula (I) and then optionally converting the resulting compound corresponding to formula (I) into a physiologically acceptable acid addition salt thereof,
or according to alternative D comprising the steps of:
(d-I) reacting a magnesium halide formed from a compound according to formula (D-I): 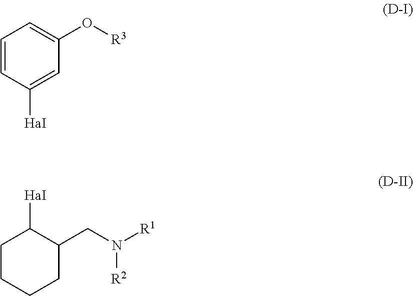
wherein Hal is a halogen atom and R³ has the above defined meaning,
with a compound according to formula (D-II), wherein Hal is a halogen atom and wherein R¹ and R² have the above defined meanings,
to a compound according to formula (I), wherein R¹, R² and R³ have the above defined meanings, and
(d-II) optionally converting the compound corresponding to formula (I) into a physiologically acceptable acid addition salt thereof.

2. A process as claimed in claim 1, wherein said compound corresponding to formula (I) is in the form of an isolated stereoisomer.

3. A process as claimed in claim 2, wherein said compound corresponding to formula (I) is in the form of an isolated enantiomer or diastereomer.

4. A process as claimed in claim 1, wherein said compound corresponding to formula (I) is in the form of a mixture of stereoisomers in any mixing ratio.

5. A process as claimed in claim 4, wherein said mixture is a mixture of enantiomers or diastereomers.

6. A process as claimed in claim 4, wherein said mixture is a racemic mixture.

7. A process as claimed in claim 1, according to alternative A, wherein
the hydrogenating step (a-I) is effected via heterogeneous or homogeneous catalysis in the presence of hydrogen, and
the reducing step (a-II) is carried out by employing at least one metal hydride, at least one borane or hydrogen in combination with a catalyst as reducing agent.

8. A process as claimed in claim 1, according to alternative A, wherein:
the hydrogenating step (a-I) is effected via heterogeneous catalysis by employing at least one catalyst selected from the group consisting of Raney nickel, palladium, palladium on carbon, platinum, platinum on carbon, ruthenium on carbon and rhodium on carbon, and
the reducing step (a-II) is carried out by employing at least one metal hydride selected from the group consisting of lithium aluminium hydride sodium borohydride, diisobutyl aluminium hydride, and selectrides as a reducing agent.

9. A process as claimed in claim 1, according to alternative A, further comprising a step (a-IV), wherein a compound corresponding to formula (A-0): 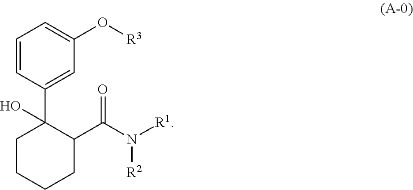
wherein R¹, R² and R³ are each independently selected from the group consisting of H and C₁₋₄-aliphatic groups
is subjected to a dehydration reaction to obtain the compound according to formula (A-I).

10. A process as claimed in claim 1, according to alternative B, wherein
in step (b-I) the compound corresponding to formula (B-I) is reacted with
R⁴—C(═S)-Hal,
wherein Hal is selected from the group consisting of Cl and Br, and R⁴ has the meaning defined in claim 1, and
in step (b-II) the conversion of the compound corresponding to formula (B-II) into a compound corresponding to formula (I) is performed in the presence of at least one radical forming agent.

11. A process as claimed in claim 1, according to alternative C, wherein the hydrogenating step (c-I) is effected via heterogeneous or homogeneous catalysis in the presence of hydrogen.

12. A process as claimed in claim 11, wherein the hydrogenating step (c-I) is effected via heterogeneous catalysis in the presence of hydrogen by employing at least one catalyst selected from the group consisting of Raney nickel, palladium, palladium on carbon, platinum, platinum on carbon, ruthenium on carbon and rhodium on carbon.

13. A process as claimed in claim 1, according to alternative C, further comprising:
a step (c-IV) wherein a compound corresponding to formula (C-0-1): 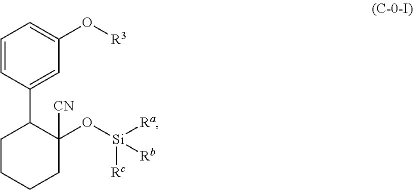
wherein
R³ is selected from the group consisting of H and C₁₋₄-aliphatic groups, and
R^{a}, R^{b} and R^{c} are each independently selected from the group consisting of C₁₋₈-aliphatic residues and aryl,
is subjected to a desilylation reaction to yield a compound corresponding to formula (C-0-II): 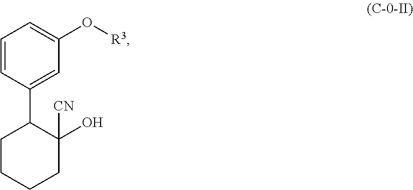
wherein R³ has the above defined meaning, and
a step (c-V) wherein the compound of formula (C-0-II) from step (c-IV) is subjected to a dehydration reaction to yield the compound corresponding to formula (C-I).

14. A process as claimed in claim 1, according to alternative D, wherein in step (d-I) the reaction of the compound corresponding to formula (D-II) with the compound formed from reacting a magnesium halide with a compound corresponding to formula (D-I) is effected via transition metal cataylsis.

15. A process as claimed in claim 1, wherein R¹ and R² and R³ are each independently selected from the group consisting of H, methyl, ethyl, n-propyl, isopropyl, n-butyl, sec.-butyl, isobutyl, and tert.-butyl.

16. A process as claimed in claim 15, wherein R¹ and R² are each independently selected from the group consisting of H and methyl, and R³ denotes H.

17. A process as claimed in claim 1, for preparing a compound corresponding to formula (Ib) 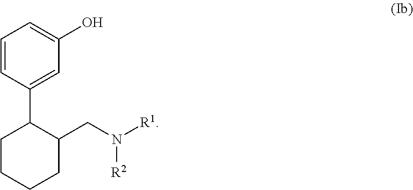
wherein R¹ and R² are each independently selected from the group consisting of H and C₁₄-aliphatic groups,
or a physiologically acceptable acid addition salt thereof.

18. A process as claimed in claim 17, wherein:
the process comprises a deprotection step of deprotecting a compound corresponding to a formula selected from the group consisting of formulas (A-0), (A-I), (A-II), (B-I), (B-II) (C-0-I), (C-0-II), (CI), (I) and (Ia), wherein R³ in each case is does not denote hydrogen to obtain a compound corresponding to formula (Ib), and
optionally converting the resulting compound corresponding to formula (Ib) into a physiologically acceptable acid addition salt thereof.
